(19) United States
(12) Patent Application Publication
(10) Pub. No.: US 2026/0363877 A1
(43) Pub. Date: Nov. 19, 2026

(54) DEVICES AND METHODS FOR SPINAL IMPLANTATION

(71) Applicant: Samy Abdou, San Diego, CA (US)

(72) Inventors: Samy Abdou, San Diego, CA (US); Brian Bowman, Carlsbad, CA (US); Colin Murphy, San Diego, VA (US)

(21) Appl. No.: 19/363,027

(22) Filed: Oct. 20, 2025

Related U.S. Application Data
(62) Division of application No. 17/352,051, filed on Jun. 18, 2021, now Pat. No. 12,447,028.
Application No. 17/352,051 is a continuation of application No. 16/590,300, filed on Oct. 1, 2019, now Pat. No. 11,179,248.
(60) Provisional application No. 62/766,127, filed on Oct. 2, 2018.
(60) Provisional application No. 62/766,123, filed on Oct. 2, 2018.

(51) Int. Cl. A61F 2/46; A61F 2/30; A61F 2/44

(52) U.S. Cl. CPC A61F 2/4611; A61F 2/4425; A61F 2002/30153; A61F 2002/30263; A61F 2/4603; A61F 2002/4615

(57) ABSTRACT

Methods and apparatus for providing correction of one or more maladies or conditions of the spinal column of a living being. In one embodiment, the apparatus includes an implant delivery instrument and an implant for use therewith. In one variant, the implant delivery instrument includes a non-detachable distraction member for distraction of the disc space and a track for slidable delivery of an implant to the distracted disc space. In another variant, the implant delivery instrument includes a detachable distraction member for distraction of the disc space and a track for slidable delivery of an implant to the distracted disc space. In the latter variant, the distraction member is co-implanted in the disc space with the implant.

### PRIORITY

This application claims the benefit of and priority to each of co-owned and co-pending U.S. Provisional Patent Application Ser. No. 62/766,127 entitled “Spinal Implant with Placement Instrument Comprising a Non-detachable Distraction Member,” filed Oct. 2, 2018, and U.S. Provisional Patent Application Ser. No. 62/766,123 entitled “Spinal Implant with Placement Instrument Comprising a Detachable Distraction Member,” filed Oct. 2, 2018, each of which is incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND

### 1. Field of the Disclosure

This disclosure relates generally to medical devices, and in one exemplary aspect to bone implants and implantation apparatus and systems, components thereof, and methods of implant placement, which can be used to, inter alia, adjust, align and maintain the spatial relationship(s) of adjacent bones or bony fragments during and/or after surgical reconstruction of skeletal segments.

### 2. Description of Related Technology

Whether from congenital malformation, degenerative disease, traumatic disruption, infection or neoplastic invasion, alteration in the anatomical alignment between the spinal vertebrae can cause significant pain, deformity, neurological decline and disability. Spinal disease is a major health problem in the industrialized world and the surgical treatment of spinal pathology is an evolving discipline. The traditional surgical treatment of abnormal vertebral motion and/or formation is the complete immobilization and bony fusion of the involved spinal segment and an extensive array of surgical techniques and implantable devices have been formulated to accomplish the treatment objective.

Vertebral fusion may be accomplished by using various approaches to a target spinal segment, such as e.g., anterior, antero-lateral, lateral, posterolateral, or posterior approaches (or variations and/or combinations thereof), each of which may have advantages and disadvantages. Frequently, circumferential fusion of the unstable or diseased portion of the spine with fixation of both the anterior and the posterior aspects thereof is desired. Such fusion typically requires that a patient undergoes a combination of the aforementioned approaches. For example, the anterior and/or the lateral approaches can be used to insert the bone graft into the disc space between the adjacent vertebrae, while the posterior approach is used to place bone screws or similar fasteners that are used to immobilize the vertebral bodies.

However, the combined surgical approaches utilized for circumferential fusion (as well as the multiple tools required for the procedures) can cause additional recovery time and/or discomfort for the patient, as well as increase the duration and/or complexity of a surgical procedure for the practitioner. Further, the foregoing conventional implantation systems and methods may be insufficient for treatment of patients with unusual or complex spinal curvatures and maladies, which may occur in conditions such as e.g., spondylolisthesis, coronal plane deformity (such as scoliosis), sagittal plane deformity (such as alternation in segmental kyphosis or lordosis), axial translation, rotational deformity, etc.

Hence there is a salient need for alternative methods and devices for the alteration and/or correction of spinal curvature, which, inter alia, enable minimally invasive procedures (including percutaneous operations) for treatment of the aforementioned spinal conditions. Further, it is desirable that such alternative methods and devices be usable in combination with conventional implantation systems and methods.

### SUMMARY

Improved devices, systems, and methods to alter vertebral alignment and/or to otherwise treat a target spinal segment are described herein.

In one aspect, an implant delivery apparatus is disclosed. In one embodiment, the apparatus comprises a distraction member configured for distraction of a disc space and an implant track configured for slidable delivery of a substantially linear implant to the distracted disc space.

In one variant, the distraction member comprises a non-detachable distraction mechanism configured to be reversibly actuated to increase a height thereof so as to enable (i) vertebral alignment or correction of e.g., spondylolisthesis, (ii) delivery of the implant to the disc space, and (iii) removal of the distraction member from the disc space after delivery of the implant. Further, the non-detachable distraction mechanism comprises an upper plate and lower plate, and the upper and lower plates are configured to be reversible movable between a closed (non-distracted) configuration and an open (distracted) configuration.

In one implementation, the upper and lower plates are configured to move along a vertical axis which is substantially perpendicular to a longitudinal axis of the implant track. Further, the upper and lower plates are configured to alter vertebral alignment in the coronal plane and reduce e.g., lateral spondylolisthesis.

In another implementation, the upper and lower plates are configured to move along a vertical axis which is substantially perpendicularly to a longitudinal axis of the implant track, as well as along a lateral axis which is substantially parallel to the longitudinal axis of the implant track. Further, the upper and lower plates are configured to increase a height of the vertebral disc space, as well as alter vertebral alignment in the sagittal plane and thereby reduce e.g., anterior or posterior spondylolisthesis.

In another variant, the distraction member comprises a detachable distraction mechanism configured to be actuated to increase a height thereof enable (i) vertebral alignment and/or correction of e.g., spondylolisthesis, (ii) delivery of the implant to the disc space, and (iii) co-implantation of the distraction member within disc space along with of the implant (after withdrawal of the implant delivery instrument).

In another embodiment, the device comprises a distraction member configured for distraction of a disc space and an implant track configured for slidable delivery of a substantially curved implant to the distracted disc space.

In another aspect, a method of inserting an implantable device within an intervertebral space is disclosed. In one embodiment, the method includes (i) inserting a distraction member of an implant delivery apparatus into an intervertebral disc space, (ii) actuating the distraction mechanism to move the distraction mechanism into an open (distracted) position and to alter a position of a superior vertebral bone relative to an inferior vertebral bone, (iii) loading an implant onto an implant track of the implant delivery apparatus, and (iv) releasing a pusher device to slidably advance the implant down the implant track and into the intervertebral disc space.

In one variant, the method further includes moving the distraction member into a closed (non-distracted) configuration and withdrawing the distraction member and the implant track from the intervertebral disc space.

In another variant, the method further includes maintaining the distraction member in the distracted configuration, detaching the distraction member from implant track, and withdrawing the implant track from the intervertebral disc space.

In another aspect, an implant is disclosed. In one embodiment, the implant comprises a superior member, an inferior member, and at least one side wall connecting the superior and inferior members. Further, superior and inferior members and the at least one side wall define an interior cavity of the implant, which is configured to (i) receive and engage with an implant track of an implant delivery apparatus during implant delivery, and (ii) receive a bone forming material after implantation thereof in the disc space.

In one variant, the implant has a generally curved cuboid shape.

In another variant, the implant has a generally rectangular cuboid shape.

In yet another variant, the superior and inferior members each comprise an inclined surface such that the implant has a greater height one one side of the implant relative to an opposing side of the implant, such that the implant has a generally tapered shape.

In another aspect, an implant delivery apparatus is disclosed. In one embodiment, the implant delivery apparatus includes an implant track that is continuous with a distraction member disposed at a distal end of the apparatus. The implant delivery apparatus enables distraction of the target intervertebral disc space and delivery of an implant using a single device.

In another aspect, a method for correction of spinal conditions is disclosed.

In a further aspect, a system for correction of spinal conditions is disclosed. In one embodiment, the system includes: (i) an implant delivery apparatus comprising a distraction member and an implant track, and (ii) an implant.

### BRIEF DESCRIPTION OF THE DRAWINGS

FIGS. 1A-1C show diagrammatic representations of a spinal vertebral bone in multiple views.

FIGS. 2A and 2B illustrate a functional spinal unit (FSU), which includes two adjacent vertebrae and the intervertebral disc between them in multiple views.

FIG. 3 illustrates a top view of a schematic illustration of a vertebral bone.

FIGS. 4A and 4B illustrate side views of a normally aligned spinal segment and a spinal segment having anterior spondylolisthesis, respectively.

FIG. 5 illustrates schematic side view of a spinal segment having an exemplary implant inserted within a disc space thereof.

FIGS. 6A and 6B illustrate views of a spinal segment showing resection of a portion of the spine.

FIG. 7A-7C respectively show top perspective, top plan and side elevation views of an exemplary implant delivery instrument according to one embodiment of the present disclosure.

FIGS. 8A and 8B show cross-sectional views of the exemplary implant delivery instrument of FIGS. 7A-7C.

FIGS. 9A and 9B show side elevation views of a first embodiment of a distraction member for use with the implant delivery instrument of FIGS. 7A-7C.

FIGS. 9C-9F show side elevation views of the first embodiment of a distraction member of FIGS. 9A and 9B, illustrating exemplary movements thereof.

FIG. 10 shows a side elevation view of a second embodiment of a distraction member for use with the implant delivery instrument of FIGS. 7A-7C.

FIGS. 11A and 11B show side elevation views of the second embodiment of a distraction member of FIG. 10, illustrating exemplary movements thereof.

FIGS. 12A-12G respectively show front and side perspective views, as well as side elevation, front elevation, top plan, and rear elevation views of a first embodiment of an implant for use with the implant delivery instrument of FIGS. 7A-7C.

FIGS. 12H and 12I show front perspective views of the implant of FIG. 12A 12G further including a movable member.

FIGS. 13-14C show top plan views illustrating loading and movement of the implant of FIGS. 12A-12G when utilized with the the implant delivery instrument of FIGS. 7A-7C.

FIG. 15 illustrates a perspective view of an exemplary articulated frame configured for use with the implant delivery instruments disclosed herein.

FIGS. 16A-16F show side and top views of a method of use of the implant delivery instrument of FIGS. 7A-7C having the first embodiment of a distraction member of FIGS. 9A and 9B for delivery of the implant of FIGS. 12A-12G.

FIGS. 17A-17D illustrate top, rear, first side and second side views of a target disc space having the implant of FIGS. 12A-12G implanted therein.

FIGS. 18A-18F show side and top views of a method of use of the the implant delivery instrument of FIGS. 7A-7C having the second embodiment of a distraction member of FIGS. 10-11B for delivery of the implant of FIGS. 12A-12G.

FIGS. 19A and 19B show schematic side and front views of a target disc space having two implants implanted therein.

FIGS. 20A and 20B are respectively perspective and cross-sectional views of an exemplary pedicle screw assembly configured for use with the implants and implantation delivery devices disclosed herein.

FIGS. 20C-20E respectively show schematic top, rear and side views of a spinal segment having the pedicle screw assembly of FIGS. 20A and 20B and the implant of FIGS. 12A-12G implanted therein.

FIGS. 21A-21D respectively show front perspective, front elevation, top plan and side elevation views of an exemplary spinous process fixation device configured for use with the implants and implantation delivery devices disclosed herein.

FIGS. 21E and 21F respectively show schematic side and top views of a spinal segment having the spinous process fixation device of FIGS. 21A-21D and the implant of FIGS. 12A-12G implanted therein.

FIGS. 22A-22C respectively show top perspective, top plan and side elevation views of an exemplary implant delivery instrument according to another embodiment of the present disclosure.

FIGS. 22E and 22E show cross-sectional views of the exemplary implant delivery instrument of FIGS. FIG. 22A-22C.

FIGS. 23A-23D respectively show front elevation, side elevation, top plan and top perspective views of a first embodiment of an implant for use with the implant delivery instrument of FIGS. 22A-22C.

FIGS. 24A-24D show top plan views illustrating loading and movement of the implant of FIGS. 22A-22C when utilized with the implant delivery instrument of FIGS. 23A-23D.

FIGS. 24E and 24F show top views of a method of use of the implant delivery instrument of FIGS. 22A-22C for delivery of the implant of FIGS. 23A-23D.

FIGS. 25A-25D respectively show top plan, front elevation, side elevation and top perspective views of a second embodiment of an implant for use with the implant delivery instrument of FIGS. 22A-22C.

FIGS. 26A-26D show top views of a method of use of the implant delivery instrument of FIGS. 22A-22C for delivery of the implant of FIGS. 25A-25D.

All Figures @ Copyright 2013-2017. Samy Abdou. All rights reserved.

### DETAILED DESCRIPTION

In order to promote an understanding of the principals of the disclosure, reference is made to the drawings and the embodiments illustrated therein, and wherein like numerals refer to like parts throughout. Nevertheless, it will be understood that the drawings are illustrative and no limitation of the scope of the claims is thereby intended. Any such alterations and further modifications in the illustrated embodiments, and any such further applications of the principles of the disclosed devices as illustrated herein are contemplated as would normally occur to one of ordinary skill in the art.

### Overview

In one aspect, improved devices, systems, and methods to alter vertebral alignment, or otherwise manipulate and fix a position of the vertebrae are described herein. Specifically, implantable devices and systems for implantation thereof (e.g., related components) and methods of use are disclosed herein.

It will be appreciated that in a variety of disorders, the vertebral bones of a human (or other vertebrate organism) may become mal-aligned and produce, among other conditions, translational, rotational and/or angulational deformities of the spinal column. The devices and methods disclosed herein can advantageously be used in the treatment of many spinal disorders, such as, inter alia, spondylolisthesis (anterior, posterior or lateral), coronal plane deformity (such as scoliosis), sagittal plane deformity (such as alternation in segmental kyphosis or lordosis), axial translation, rotational deformity, and the like.

In one example, spinal segment to be surgically treated using the methods and apparatus disclosed herein includes at least a superior vertebral bone, an immediately inferior vertebral bone, and the intervening intervertebral disc space. A spinal segment comprised of two immediately adjacent vertebral bones and the intervertebral disc space disposed therebetween defines a “functional spinal unit” (FSU)—as described further below. An FSU to be surgically treated will be referred to as a target FSU and its intervertebral disc space as a target intervertebral disc space.

In one embodiment, an implant delivery apparatus includes a body, a handle extending from the body, an implant track disposed on distal side of the body, a distraction mechanism disposed at a distal end of the implant track, an implant pusher device disposed on operatively connected to the body. The implant delivery apparatus is configured to enable distraction of the target intervertebral disc space and delivery of an implant to the distracted disc space. The distraction mechanism is configured to move the vertebral bones of the target FSU into a desired configuration and correct alignment thereof. The implant track is configured to have an implant loaded thereon and slidably delivered to the distracted disc space utilizing the implant pusher. The implant includes a superior plate (configured to abut the superior vertebral bone), an inferior plate (configured to abut the inferior vertebral bone), and a side wall connecting the superior plate and the inferior plate. The superior plate, the inferior plate, and the side wall define a cavity which is configured to receive an outer aspect of the implant track. Further, protrusions may be disposed on the interior surfaces of the plates, which are configured to engage with grooves on the implant track. Alternatively or additionally, protrusions/ridges may be disposed on the exterior surface of the implant track, which are configured to engage with grooves on the interior surfaces of the implant.

In one variant, the distraction member is non-detachable and is configured to be removed from the target disc space after implantation of the implant. In another variant, the distraction member is detachable and is configured to be released from the implant track and implanted in the disc space along with the implant (having been advanced down the implant track and into the target disc space).

In one embodiment, a method of treatment includes entering the target intervertebral disc space and removing at least a portion of the viscoelastic material that comprises the natural nucleus pulposus within (at least a portion of) the intervertebral disc space. The target intervertebral disc space may be accessed using various surgical approaches (such as e.g., a direct anterior approach, an anterolateral approach, and/or a direct lateral approach, posterolateral approach, posterior approach, etc.), thereby creating one or more operative corridors at desired vertebral level(s) of the spinal column.

After removal of the viscoelastic material (and optional decortication of the bony surfaces adjacent to the evacuated disc space segment(s)), the method further includes inserting the distraction member into the target intervertebral disc space, and actuating the distraction member to alter alignment of the superior and inferior vertebral bones. Next, an implant is loaded onto the implant track and advanced down the track via release of the implant pusher, and the implant is positioned with the target intervertebral disc space. In one variant, the distraction member is removed from the disc space as the implant delivery instrument is withdrawn. In another variant, the method further includes detaching the distraction member from the implant track such that the distraction member is implanted into the disc space along with the implant.

### Detailed Description of the Exemplary Embodiments

Exemplary embodiments of the apparatus and methods of the present disclosure are now described in detail.

It will be appreciated that while the exemplary embodiments are described with respect to human beings, various of the methods, apparatus and systems disclosed herein may be applied to other species having a spinal structure (i.e., vertebrates).

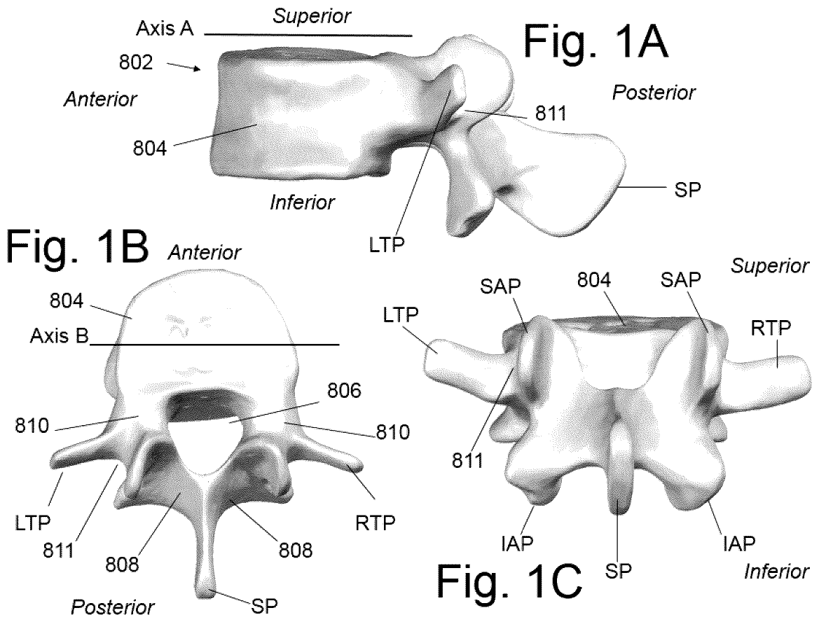

FIGS. 1A-1C show diagrammatic representations of a spinal vertebral bone 802 in multiple views. For clarity of illustration, the vertebral bone of FIGS. 1A-1C and those of other illustrations disclosed herein are represented schematically and it should be appreciated that actual vertebral bodies may include anatomical details that are not shown in these figures. Further, it is understood that the vertebral bones at a given level of the spinal column of a human or animal subject will contain anatomical features that may not be present at other levels of the same spinal column. The illustrated vertebral bones are intended to generically represent vertebral bones at any spinal level without limitation. It will be appreciated that the disclosed devices and methods may be employed at any applicable spinal level.

Additionally, the term “sagittal plane”, as used herein, refers without limitation to the plane that splits the body into left and right segments. The terms “mid-sagittal plane” or “median plane”, as used herein, refer to the plane that specifically splits the body into equal left and right halves. The term “coronal plane”, as used herein, refers without limitation to the plane that divides the body into anterior (front) and posterior (back) segments. It will be appreciated that the coronal and sagittal planes are substantially perpendicular to one another.

As can be seen in FIGS. 1A-1C, the vertebral bone 802 contains an anteriorly-disposed vertebral body 804, a centrally-disposed spinal canal 806 and a posteriorly-placed lamina 808. The pedicle segments 810 of the vertebral bone 802 form the lateral aspects of the spinal canal 806 and connect the laminas 808 to the vertebral body 804. The spinal canal 806 contains neural structures such as the spinal cord and/or nerves. A midline protrusion termed the spinous process (SP) extends posteriorly from the medial aspect of laminas 808. A protrusion extends laterally from each side of the posterior aspect of the vertebral bone 802 and is termed the transverse process (TP). A right transverse process (RTP) extends to the right from the lateral aspect of the right pedicle. A left transverse process (LTP) extends to the left from the lateral aspect of the left pedicle. A superior protrusion extends above the lamina 808 on each side of the vertebral midline, and is termed the superior articulating process (SAP). An inferior protrusion extends inferiorly below the lamina 808 on each side of the vertebral midline, and is termed the inferior articulating process (IAP).

As a brief aside, it is noted that the posterior aspect of the pedicle 810 can be accessed at an indentation 811 in the vertebral bone 802 between the lateral aspect of the SAP and the medial aspect of the TP. In surgery, it can be common practice to anchor a bone fastener into the pedicle portion 810 of a vertebral bone 802 by inserting the fastener through indentation 811 and into the underlying pedicle 810 in a posterior to anterior direction.

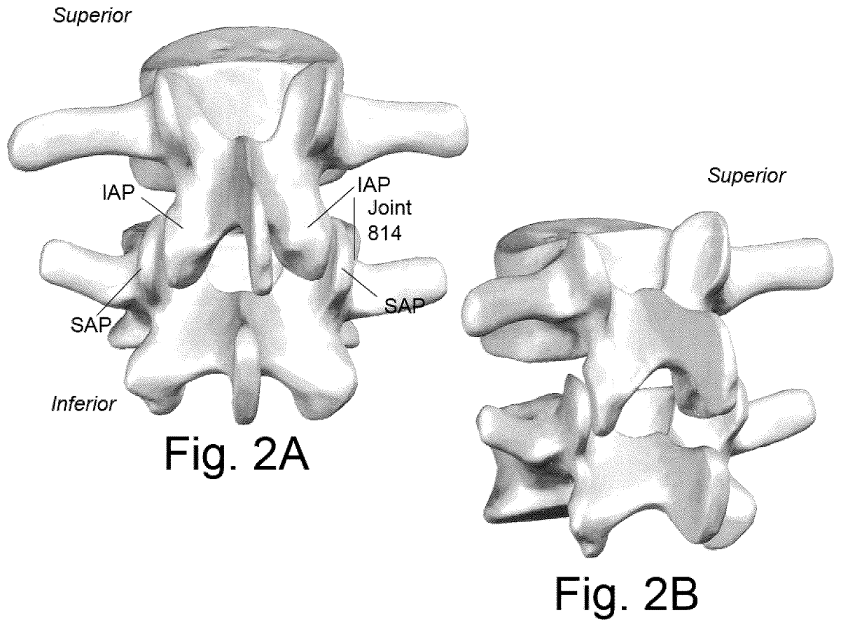

FIGS. 2A and 2B illustrate a functional spinal unit (FSU), which includes two adjacent vertebrae and the intervertebral disc disposed therebetween. The intervertebral disc resides between the inferior surface of the upper vertebral body and the superior surface of the lower vertebral body, although it is not specifically shown in the figures. FIG. 2A shows the posterior surface of the adjacent vertebrae and the articulations between them. FIG. 2B shows an oblique view. The FSU contains three joints between the two vertebral bones, with the intervertebral disc comprising the anterior joint. The posterior joints include a facet joint 814 on each side of the midline, wherein each facet joint 814 is comprised of the articulation between the IAP of the superior vertebral bone and the SAP of the inferior bone.

These illustrations and definitions of anatomical structures are known to those of ordinary skill in the art. They are described in more detail in Atlas of Human Anatomy, by Frank Netter, third edition, Icon Learning Systems, Teterboro, New Jersey, which is incorporated herein by reference in its entirety. It should be appreciated that the directional language and terms regarding orientation such as upper, lower, upward, downward etc., are used throughout merely for convenience of description and are not limiting.

As shown in FIG. 3, the apophyseal ring is an outer rim segment of the vertebral body that is located on each of the superior and the inferior surfaces of a vertebral bone and comprise bony surfaces that abut the intervertebral discs. Further, the apophyseal ring is circumferentially positioned and forms the most dense and strongest portion of the superior and inferior surfaces of said vertebral bone. The ring is comprised of dense bone that anchors the external fibers of the annulus fibrosis of the adjacent intervertebral disc. The apophyseal ring is discussed in greater detail in The epiphyseal ring: a long forgotten anatomical structure with significant physiological function. Dar G, et al. Spine (Phila Pa 1976). 2011 May 15; 36(11):850-6, which is incorporated herein by reference in its entirety.

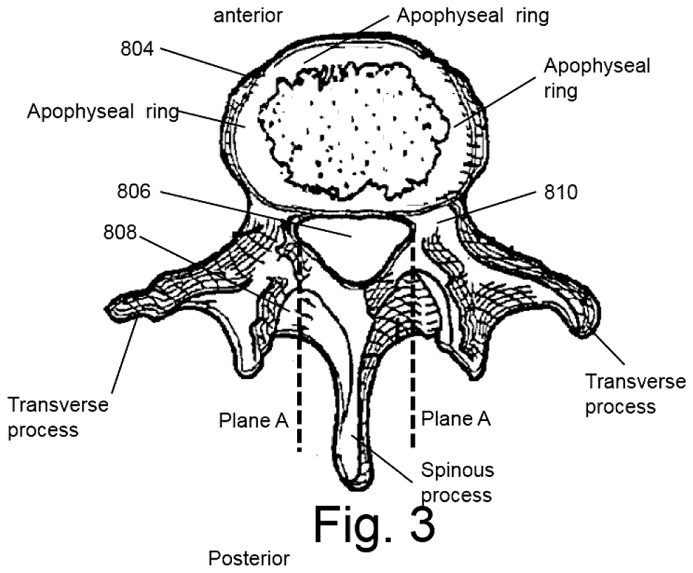

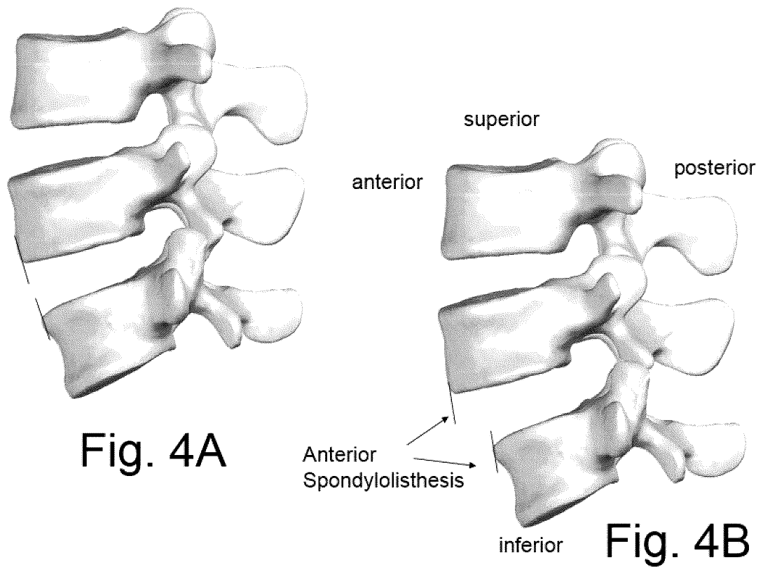

In a healthy spine within normal physiological parameters (such as that shown in FIG. 4A), the two facet joints of a functional spinal unit (FSU) collectively function to prevent, inter alia, aberrant movement in the horizontal (i.e., axial) plane of the superior vertebral bone relative to the inferior vertebral bone. As a brief aside, it is noted that the horizontal plane of a human spine refers to a plane of the erect spine that is substantially parallel to a level floor on which the subject is standing.

However, with aging and spinal degeneration, displacement of the vertebral bones in the horizontal plane may occur, which is a condition termed spondylolisthesis. FIG. 4A illustrates three vertebral bones with relatively normal alignment, whereas FIG. 4B shows the anterior displacement of the middle bone relative to the inferior-most bone. As illustrated therein, the vertebral column of FIG. 4B may be characterized as having an anterior spondylolisthesis of the middle vertebral bone relative to the inferior-most vertebral bone. A spondylolisthesis can be anterior, as shown in FIG. 4B, posterior (where a superior vertebral bone of an FSU is posteriorly displaced in the horizontal plane relative to the inferior vertebral bone) or lateral. In general, anterior sponylolisthesis is more clinically relevant than posterior spondylolisthesis, and any of the foregoing types of sponylolisthesis can be further classified based on the extent of vertebral displacement. Characterization of spondylolisthesis is discussed in greater detail in Principles and Practice of Spine Surgery by Vaccaro, Bets, Zeidman; Mosby press, Philadelphia, PA; 2003, which is incorporated herein by reference in its entirety.

With degeneration of the spine, constriction of the spinal canal and impingement of the nerve elements contained therein frequently occurs, and is termed spinal stenosis. Spondylolisthesis can exacerbate the extent of nerve compression within the spinal canal as misalignment of bone within the horizontal plane will often further reduce the size of the spinal canal. Relief of the compressed nerves can be achieved by the surgical removal of the bone and ligamentous structures that constrict the spinal canal. Decompression of the spinal canal can, however, further weaken the facet joints and increase the possibility of additional aberrant vertebral movement. Therefore, conventional spinal decompression procedures may actually worsen the extent of spondylolisthesis or produce spondylolisthesis in an otherwise normally aligned FSU elsewhere in subject's the spine. Accordingly, after decompression, surgeons will commonly fuse and immobilize the adjacent spinal bones in order to prevent the development of post-operative vertebral misalignment and/or spondylolisthesis.

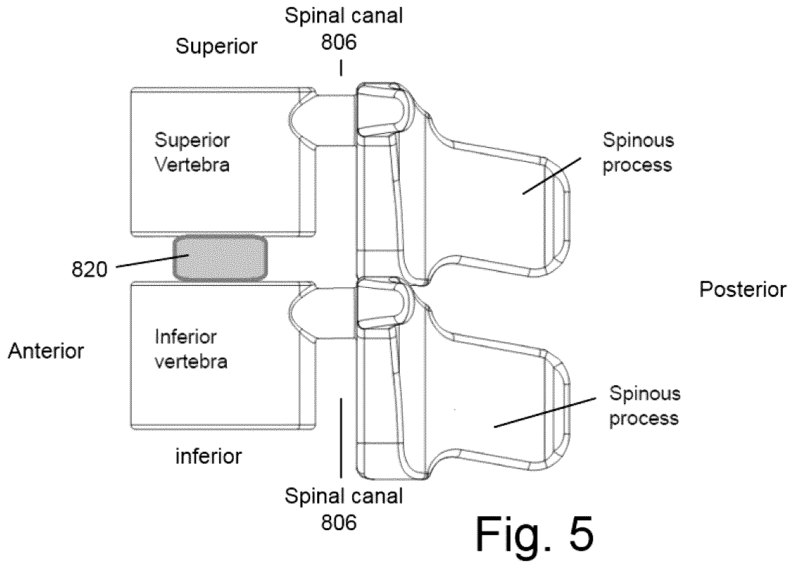

Regardless of the clinical reasoning or indication for fusion of the vertebral bones, many surgeons position an implant within the disc space that rests between the two vertebral bones which are to be fused. An example of a generic interbody implant 820 positioned within a disc space between superior and inferior vertebral bodies of an immobilized FSU is depicted in FIG. 5, where a side view of a schematic FSU is shown. Many embodiments of interbody implants are known in the art, such as those shown and described in U.S. Pat. Nos. 4,636,217; 5,015,247; 5,192,327; 5,443514; 5,749,916, 6,251,140; 6,342,074; 6,706,070; 6,767,367; 6,770,096; 6,852,127; 7,037,339; 7,227,477; 7,641,690, each of which is incorporated herein by reference in its entirety.

In general, an interbody implant is usually comprised of an outer superstructure manufactured from a synthetic biocompatible material (such as e.g., metal alloy, plastic material, ceramics, or the like), and an internal cavity contained therein. The internal cavity is configured to receive and house a bone forming material that may be inserted by the surgeon into the interbody implant at the time of implantation. Openings in the superstructure permit communication and fusion between the vertebral bone(s) outside of the device and the bone forming material contained within cavity. In general, the superstructure separates and supports the vertebral bones that abut the implanted disc space. In this way, the device can be used to maintain the disc space height. The internal cavity contains the bone formation material that will form a fusion mass which will (over time) extend from the superior vertebral bone to the inferior vertebral bone. When the superstructure is manufactured from metallic alloy, it can be advantageously made of limited thickness thereby providing a larger internal cavity for containment of a greater volume of bone forming material. However, the metallic superstructure is generally X-ray opaque, thereby limiting the ability to follow bone healing in the post-operative period via X-ray imaging. In contrast, manufacture of the superstructure from plastic materials (such as PEEK or the like) or ceramic permits X-ray visualization of the healing bone within, but significantly limits the size of internal cavity and the volume of bone forming material contained therein as the foregoing materials have a lower mechanical strength (relative to metallic materials) and require thicker walls to provide the necessary resistance to load, strain and/or stress on the implanted device.

In alternate embodiments, interbody implants may be manufactured without a dedicated internal cavity. In these latter embodiments, the outer surface of the implant may be at least partially comprised of a material capable of promoting osseointegration (e.g., direct bony ingrowth into the implant from the adjacent bone).

Considerable clinical experience has been gained by the implantation of the aforedescribed interbody implants via a posterior surgical corridor, and practitioners have become aware of the limitations and disadvantages associated therewith. For example, in a first limitation, the implants are generally large, having a width of at least 9 mm, and therefore require substantial bony resection of the posterior spinal elements for implantation. Specifically, implantation of such devices through a posterior surgical approach often involves removal of substantial portions of the facet joint at the implanted level. Facet joint resection can add time and complexity to the surgical work, as well as increased pain and/or recovery time for the patient. Further, in another limitation, facet joint resection can also significantly destabilize the implanted FSU so that pedicle screw fixation is needed to re-stabilize the spine. In other words, implantation of the interbody device may require a high degree of bony resection so as to require extensive supplemental fixation, which (again) adds time and complexity to the surgical process and can also increase pain and/or recovery time for the patient. In yet another limitation, given the proximity to nerve elements to the posterior surgical corridor, implant placement with limited facet resection requires a greater degree of nerve retraction and increases the risk of nerve injury.

Prior attempts to reduce the width of the interbody implant and avoid the foregoing limitations have yielded implants with a height to width ratio that is greater than one, however, these implants have an increased risk of roll-over and/or dislodging within the disc space.

The interbody implants and associated implantation devices and methods disclosed herein address the above identified issues with conventional spinal implants and techniques. The devices and methods are particularly advantageous for use in minimally invasive procedures-including percutaneous operations. Although specific examples are shown and described herein, it will be appreciated that the spinal implantation devices and methods of implantation of the present disclosure may be employed in a myriad of applicable interbody fusion procedures using a variety of surgical corridor/approaches and at various spinal segments and/or structures.

It is a purpose of the present disclosure to describe implantation devices and methods for the safe and reproducible placement of an interbody device into an intervertebral disc space. In one embodiment, the interbody device may be employed without other bone fixation implants (i.e., as a “stand alone” device). In another embodiment, the interbody device may be employed in conjunction with a spinous process fixation implant. In yet another embodiment, the interbody device may be used with screw fixation of the vertebral bones, such as, for example, pedicle screw assemblies or the like. In one implementation, a pedicle screw assembly is placed into an ipsilateral pedicle of each of the superior and inferior vertebral bones that abut the implanted disc space. The screw assemblies are joined by an interconnecting member, such as a rod, and the screw assembly and joined interconnecting member are used to rigidly fixate the vertebral bones to one another. The interbody device or the pedicle screw/rod assembly may be used on one side (i.e., unilateral) of the vertebral midline alone or, alternatively, on both sides (i.e., bilateral) of the vertebral midline, where the vertebral midline is substantially defined by the mid-sagittal plane that bisects the implanted disc space/vertebral bones into a right half and a left half. In still other embodiments, the interbody device may be used with one or more additional bone fixation implants.

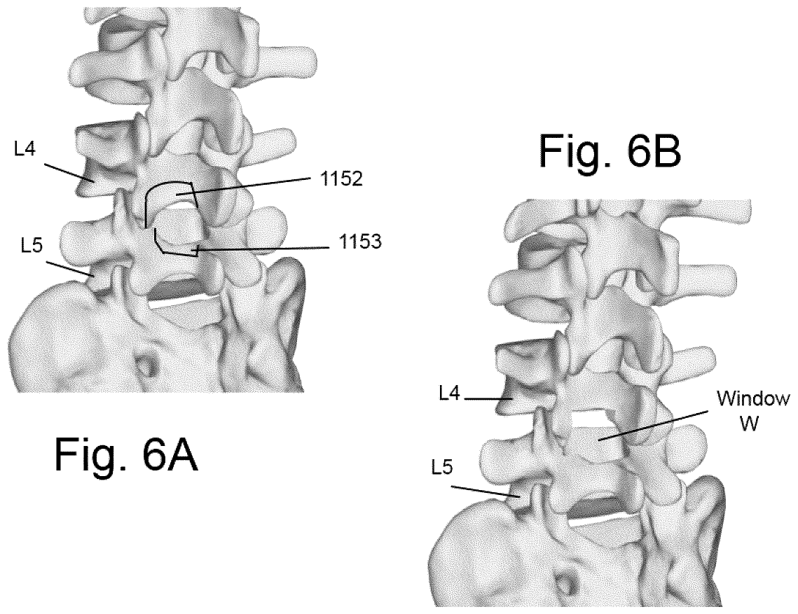

In one embodiment, the disc space that is targeted for inter-body device implantation is identified using radiographic imagining techniques (such as X-rays, CT, MRI and the like). A skin incision is made in the skin immediately posterior to the target disc space. The paraspinal muscles are retracted and a corridor is developed adjacent to the spinous process and the posterior aspect of the lamina. The lamina of each of the superior and inferior vertebrae that border the targeted disc space are identified preferably by use of an imaging modality. Resection of the lamina posterior to the target disc space is performed, where at least a portion of the inferior aspect of the lamina of the superior vertebral bone (i.e., the vertebral bone that forms the superior border of the target disc space) is removed. FIG. 6A shows a schematic depiction of an exemplary resection of a segment 1152 of the inferior aspect of the lamina of the superior vertebral bone (when targeting the L4/5 disc space).

An additional resection of the lamina posterior to the target disc is then performed, where at least a portion of the superior aspect of the lamina of the inferior vertebral bone (i.e., the vertebral bone that forms the inferior border of the target disc space) is removed. The schematic depiction of FIG. 6A additionally shows an exemplary resection of a segment 1153 of the superior aspect of the lamina of the inferior vertebral bone. At least a portion of the ligament (i.e., the ligamentum flavum) that spans the region of lamina resection can also be removed such that the posterior aspect of the thecal sac is exposed through a window W of FIG. 6B. While shown as being formed on only one side of the midline in FIG. 6A, alternatively the window W may be formed bilaterally (i.e., the window W may be formed on both sides of the vertebral midline, where the vertebral midline is defined by a sagittal plane that substantially extends through the spinous process and divides a vertebral bone into left and right halves).

The posterior aspect of the target disc space can then be exposed through a corridor that is lateral to the thecal sac. The thecal sac may be retracted gently in the medial direction to enable identification of the posterior aspect of the target disc space. The disc space can then be entered and at least a segment of the disc material may be removed (i.e., discectomy). If necessary, a collapsed disc space having a small vertical height that is substantially below the normal value for that disc space level may be distracted to a desired (increased) height via sequential or iterative placement of shims or distractors within the disc space, where the disc space height is defined as the vertical distance from the superior disc space surface to the inferior disc space surface.

Additionally or alternatively, an implant placement instrument can be configured for distraction of the disc space. For example, the exemplary implant placement instruments disclosed herein have an intra-discal segment that is sized to be positioned within the disc space. The intra-discal segment includes opposing upper and lower plates or members, and the upper plate can be forcibly distracted away from the lower plate to enable the implant placement instrument to function as distraction device. Accordingly, the vertebral bone superior to the target disc space and its immediately inferior vertebral bone can be forcibly moved away from one another, thereby increasing the vertical height of the target disc space.

### Implant and Implant Placement Apparatus

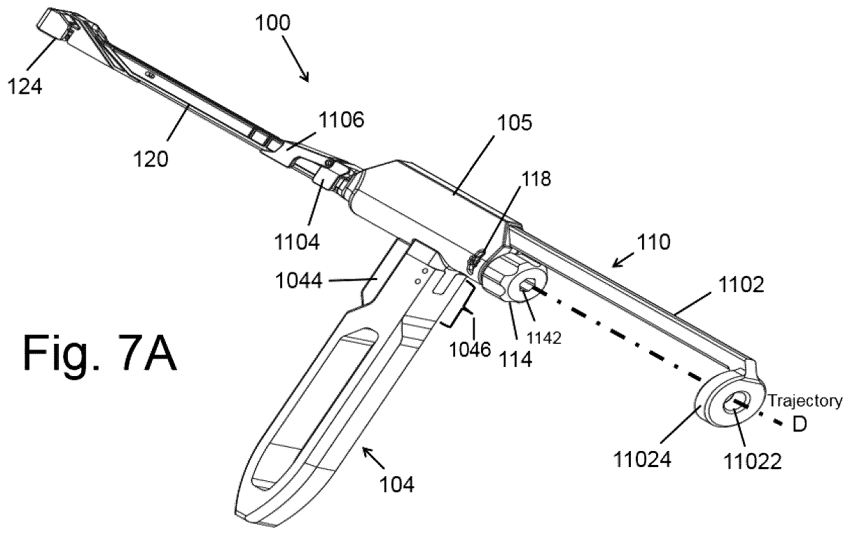

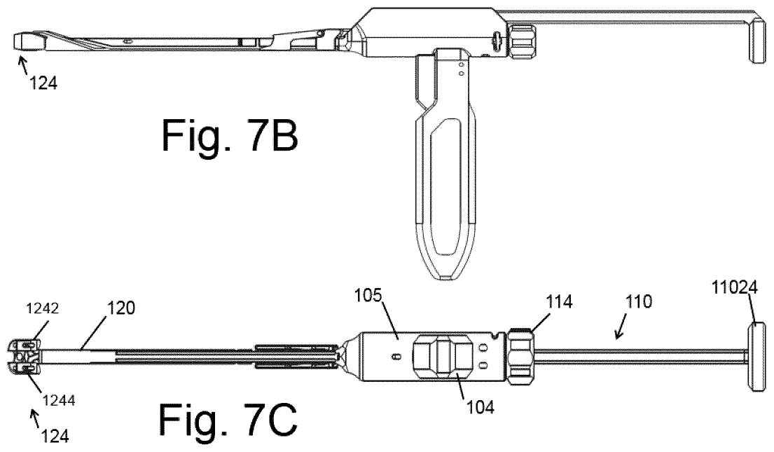

One exemplary embodiment of an implant placement instrument 100 is show in FIGS. 7A-11B. As depicted therein, the implant placement instrument 100 includes a body 105 and a handle 104 extended outwardly therefrom. An implant track 120 extends outwardly from an anterior surface of the body 105, and a distraction member extends outwardly from a distal end of the implant track 120.

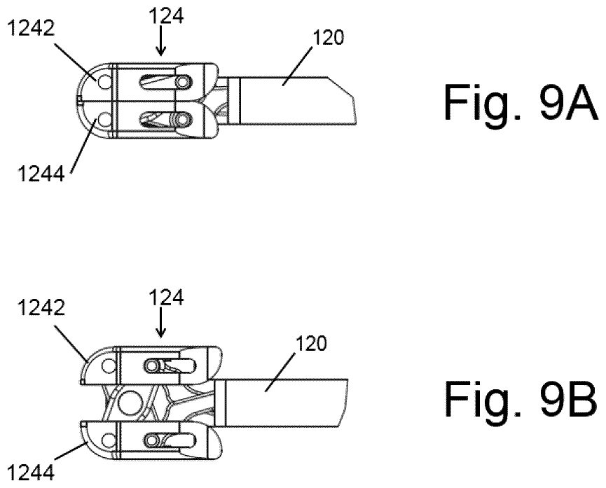

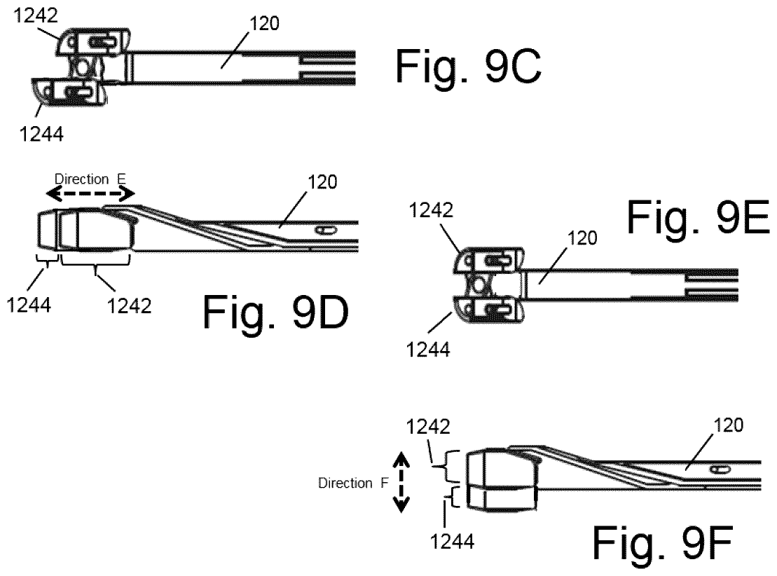

As shown in FIGS. 7C and 9A-9F, in one embodiment, a distraction member 124 includes an upper plate 1242 and a lower plate 1244. The plates 1242 and 1244 are configured to be distracted apart so as to apply a distraction force onto the vertebral bones when the distraction member 124 is positioned within the intervertebral disc space. A distal end of each of the plates 1242 and 1244 comprises a generally continuously curved surface, such that each distal end has a generally quarter circle cross-section. A proximal end of each of the plates 1242 and 1244 also comprises a curved surface, although the proximal ends are of an irregular curvature which differs from the continuous curvature of the distal ends. In alternate implementations, the curvature of the proximal and distal ends may be identical (e.g., the proximal ends may comprise a continuously curved surface), or the proximal ends may have a non-curved surface (e.g., a squared surface, an inclined surface, etc.).

An exemplary closed (non-distracted) configuration of the distraction member 124 is shown in FIG. 9A, while an exemplary open (distracted) configuration is shown in FIG. 9B. As can be seen in FIG. 9A, in the closed configuration of the present embodiment, an interior surface of the upper plate 1242 is abutted to an interior surface of the lower plate 1244, and the continuously curved distal ends of the upper and lower plates 1242 and 1244 cooperatively form a generally half circle cross-section. Further, a height of the plates in the closed configuration is greater than a height of the implant track 120 (i.e., the exterior surfaces of the plates 1242 and 1244 respectively extend above and below a boundary of the implant track 120). The half circle cross-section of the distraction member in the closed configuration (forming curved, non-blunt end of the distraction member) may assist in enabling the distraction member to be pushed through tissues and/or a tissue corridor within the subject during insertion of the distraction member into the intervertebral disc space. As can be seen in FIG. 9B, in the open configuration, a space is disposed between the interior surface of the upper plate 1242 and the interior surface of the lower plate 1244, such that an overall height of the distraction member 124 is increased in the open configuration relative to the closed configuration. It is noted that while the plates 1242 and 1244 are movable relative to one another in the present embodiment, the upper and lower surfaces of the implant track 120 are not movable relative to each other (i.e., the upper and lower surfaces of the implant track are non-distractable) and remain stationary during distraction of the distraction member and delivery of the implant to the target disc space.

In the present embodiment, the distraction member 124 is non-detachable from the implant placement instrument 100. Accordingly, the distraction member is removed the target disc space at the conclusion of the procedure, and does not remain implanted therein. As shown in the illustrated embodiment, the plates 1242 and 1244 are joined and operable via crossed linkages (e.g., a scissor jack-like distraction mechanism), which are coupled to a distal end of implant track 120 and configured for reversible distraction of the distraction member 124. It is contemplated, however, that alternative or additional mechanisms for distraction may be incorporated into the distraction member 124. These alternative/additional distraction mechanisms may include, for example, wedges/inclines, pulleys, balloons, magnets, hydraulic drives, pistons or the like.

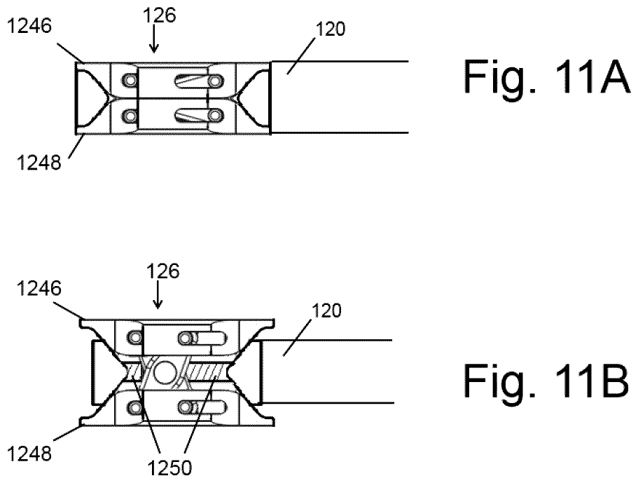

For example, in one alternate embodiment, the distraction member may comprise a worm screw drive or gear mechanism and an associated ridged track, which is configured to be turned (wound) via an attachable and/or insertable adjustment tool or the tool may be integrated into the implant placement instrument (see e.g., the distraction mechanism discussed below with reference to FIGS. 11A and 11B). The tool may be operated in a first rotational direction to increase a height of the distraction member, thereby increasing a distance between the superior and inferior members. Further, in some examples, the tool may be operated in a second rotational direction to decrease the height of the distraction member, and thereby decrease the distance between the upper and lower plates. Various exemplary mechanical (non-fluidic) mechanisms that can be adapted into the distraction member are shown and described in U.S. Pat. No. 7,909,870 and U.S. Patent Publication No. 2003/0163199, each of which is incorporated herein by reference in its entirety.

In another embodiment, the distraction member may include a balloon distraction mechanism made of either non-compliant or compliant material, which may be porous or non-porous, or may include a mesh material which may be coated or lined with a porous or non-porous material. The balloon may further include a port for coupling to a source of an inflation and/or expansion medium (e.g., a gas, a liquid, a semi-solid, a gel, a liquid that hardens into a solid material, etc.) for inflating and/or expanding the distraction mechanism. The devices can further include one or more anchoring or attachment features for fixing the balloon to one or both of the superior and inferior members. Actuation of such an embodiment of the distraction mechanism involves inflation of the balloon with the expansion medium, where the act of balloon inflation provides at least part of the force needed to produce the change in configuration of the distraction member (such as an increase in the height of the distraction member). An exemplary balloon driven distraction mechanism that can be adapted into the distraction member is shown and described in U.S. Pat. No. 8,123,807, which is incorporated herein by reference in its entirety.

In yet another embodiment, the distraction member can include a piston-based distraction mechanism. Specifically, a piston can be disposed within a cavity of one or both of the upper or lower plates. Note that in one implementation, the distraction member can include one or more apertures that allow filling and/or bleeding of the working fluid from the piston chamber. See also, e.g., U.S. Patent Application Publication No. 2007/0093901, which is incorporated herein by reference in its entirety, and describes the exemplary use of pistons in the manufacture of an expandable interbody implant, which may be integrated into the distraction member described above. Turning now to FIGS. 9C-9F, exemplary movements and orientations of the plates 1242 and 1244 relative to each other during distraction of the distraction member 124 are illustrated. In one implementation, shown in FIGS. 9C and 9D, the plates 1242 and 1244 are configured to move both (i) vertically away from one another so as to cause vertebral distraction of the disc space generally along a longitudinal axis of the spine when in erect orientation, and (ii) along a direction E, which is substantially parallel to the longitudinal axis of the implant track 120. In the closed configuration (FIG. 9D) the plates 1242 and 1244 have a side-by-side arrangement where a longitudinal axis of each of the plates is substantially aligned with the longitudinal axis of the implant track 120. In the open configuration, the plates are separated by a space, and the lower plate 1244 is positioned forward of (or distal relative to) the upper plate 1242. The present implementation (illustrated in FIGS. 9C and 9D) enables the distraction member 124 to e.g., increase a height of the vertebral disc space, as well as alter vertebral alignment in the sagittal plane and thereby reduce anterior or posterior spondylolisthesis of the target FSU. It will be appreciated that the plates can be moved in one or both of the foregoing directions to varying degrees as necessary for adjustment of the target FSU.

In another implementation, FIGS. 9E and 9F illustrate that the plates 1242 and 1244 are configured for movement along direction F, which is substantially perpendicular to the longitudinal axis of the track 120. In the closed configuration (FIG. 9F), the plates are in a stacked arrangement, and an interior surface of the upper plate 1242 is abutted to an interior surface of the lower plate 1244. Further, the longitudinal axis of the upper plate 1242 is generally aligned with the longitudinal axis of the implant track 120, whereas the longitudinal axis of lower plate 1244 is parallel to and offset from the longitudinal axis of the implant track 120. In the open configuration (FIG. 9E), the plates have a space disposed therebetween, yet remain in a generally stacked arrangement. In this latter implementation, the distraction member 124 is configured to, e.g., alter vertebral alignment in the coronal plane and reduce lateral spondylolisthesis.

While the plates 1242 and 1244 of the distraction member 124 are shown as moving into a substantially parallel configuration relative to one another (i.e., in each of the foregoing implementations shown in FIGS. 9C-9F the longitudinal axis of the upper plate 1242 remains substantially parallel to the longitudinal axis of the lower plate 1244 in both the non-distracted and the distracted configurations), in alternate or additional implementations, the plates may be configured to move into non-parallel configuration in order to impart a desired anatomical relationship to the target FSU. For example, the distraction member can be configured such that a distance between the distal ends of the plates is greater than a distance between the proximal ends of the plates when the distraction member is in the open/distracted configuration. In this implementation, the distraction member can be used to not only re-align the vertebral bones in the various planes described but to also confer a greater lordosis (and/or kyphosis) to the disc space of the target FSU.

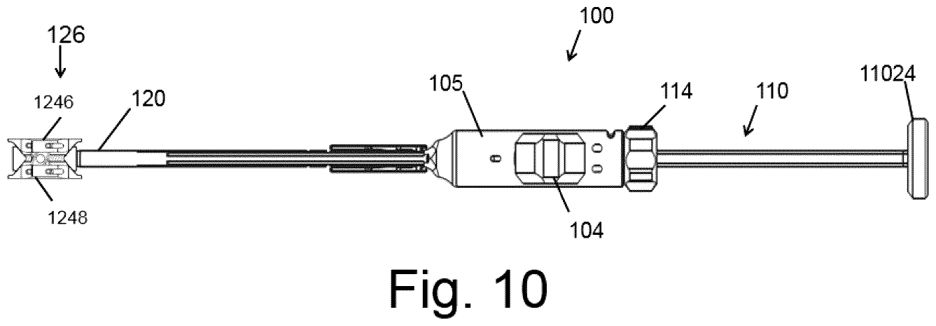

Turning now to FIGS. 10-11B, in an alternate embodiment, a detachable distraction member 126 includes an upper plate 1246 and a lower plate 1248. Similar to the plates 1242 and 1244 of the distraction member 124, the plates 1246 and 1248 are configured to be distracted apart so as to apply a distraction force onto the vertebral bones when the distraction member 126 is positioned within the intervertebral disc space. Different from the plates 1242 and 1244, each of the distal and proximal ends of the plates 1246 and 1248 comprises a generally inclined surface have an overhang at a distal tip thereof. In alternate implementations, the proximal and distal ends comprise non-inclined surfaces (e.g., each having a squared surface, a continuously curved surface, an irregularly curved surface, etc.), or the proximal and distal ends may have different configurations (e.g., the proximal ends may comprise a continuously or irregularly curved surface, while the distal ends comprise the inclined surface).

An exemplary closed (non-distracted) configuration of the distraction member 126 is shown in FIG. 11A, while an exemplary open (distracted) configuration is shown in FIG. 11B. As can be seen in FIG. 11A, in the closed configuration, an interior surface of the upper plate 1246 is abutted to an interior surface of the lower plate 1248, and the plates have a generally stacked arrangement. Further, the inclined distal and proximal ends of the upper and lower plates cooperatively form a generally “V” shaped space therebetween, which overlaps with (aligns with) the implant track 120. The relatively small height the detachable distraction member in the closed configuration may assist in enabling the distraction member to be pushed through tissues and/or a tissue corridor within the subject during insertion thereof into the intervertebral disc space.

As depicted in FIG. 11B, in the open configuration, the upper and lower plates 1246 and 1248 have a space disposed therebetween, and remain in a generally stacked arrangement. Further, the upper and lower plates 1246 and 1248 form an “X”-like configuration.

The distraction member 126 is configured to be a separate, detachable member that may be uncoupled from the implant track 120 of the implant placement instrument 100 so as to enable implantation of the distraction member within the disc space in addition to an implant that is delivered by advancement along the implant track 120. After detachment from the implant placement instrument 100, the distraction member 126 is configured to be retained (or otherwise locked) in the distracted configuration. In the present embodiment, the plates 1246 and 1248 of the distraction member 126 are illustrated as being joined by linkages and driven by a screw 1250 (similar to a scissor jack-like distraction device with a worm screw drive). Specifically, the distraction member 126 comprises a worm screw drive or gear mechanism and an associated ridged track, which is configured to be turned (wound) via an adjustment tool integrated into the implant placement instrument. The tool may be operated in a first rotational direction to increase a height of the distraction member, thereby increasing a distance between the upper and lower plates. Further, the tool may be operated in a second rotational direction to decrease the height of the distraction member, and thereby decrease the distance between the upper and lower plates. While plates 1246 and 1248 are illustrated as being joined by linkages and driven by the screw 1250, it is contemplated that alterative distraction mechanisms may incorporated into the distraction member 126 (such as, e.g., wedges/inclines, pulleys, balloons, magnets, hydraulic drives, pistons or the like, specific examples of which are discussed above).

Further, while the plates 1246 and 1248 of distraction member 126 are shown as moving into a substantially parallel configuration relative to one another (i.e., in the foregoing implementation shown in FIGS. 11A-11B the longitudinal axis of the upper plate 1246 is substantially parallel to the longitudinal axis of the lower plate 1248 in both the non-distracted and the distracted configurations), in alternate or additional implementations, the plates may be configured to move into non-parallel configuration in order to impart a desired anatomical relationship to the target FSU. For example, the distraction member can be configured such that a distance between the distal ends of the plates is greater than a distance between the proximal ends of the plates when the distraction member is in the open/distracted configuration.

During use of the implant placement instrument 100, the operator may hold and manipulate the instrument using handle 104 for advancement of the distraction member 124 or 126 into the intervertebral disc space. If needed, a mallet, or the like, may be used to hammer against a proximal end 11024 of a proximal member 1102 in order to advance the distraction member 124 or 126 into the intervertebral disc space.

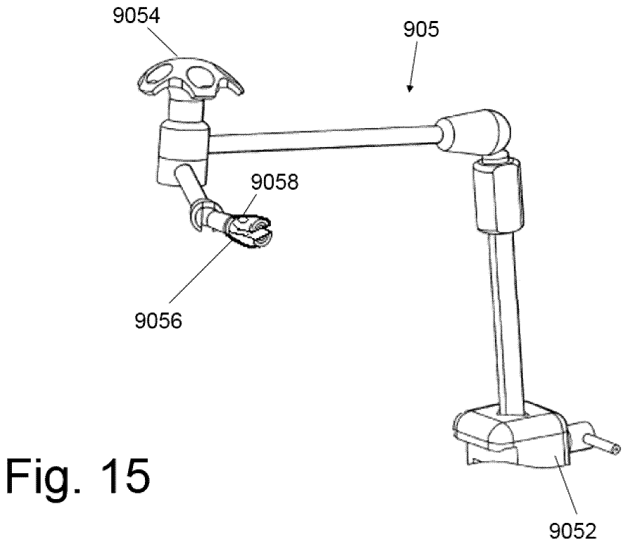

Optionally, the implant placement instrument 100 may be stabilized via attachment to a fixation assembly that is anchored to a segment of the patient, such as, for example, a bony segment of the vertebral bone or another skeletal bone via a bone screw or the like. Additionally or alternatively, the implant placement instrument 100 may be stabilized via attachment to a fixation assembly that is anchored to the operating table on which the patient is positioned for surgery. An exemplary fixation assembly 905 is shown in FIG. 15 and is discussed elsewhere herein. The implant placement instrument 100 may also comprise a coupling region (for example, a coupling region 1046 of handle 104 shown in FIG. 7A) for attachment onto one or more of the fixation assemblies.

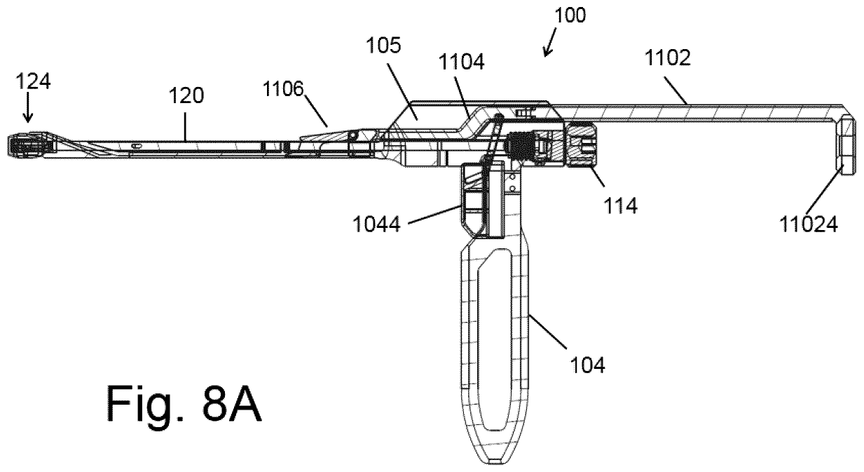

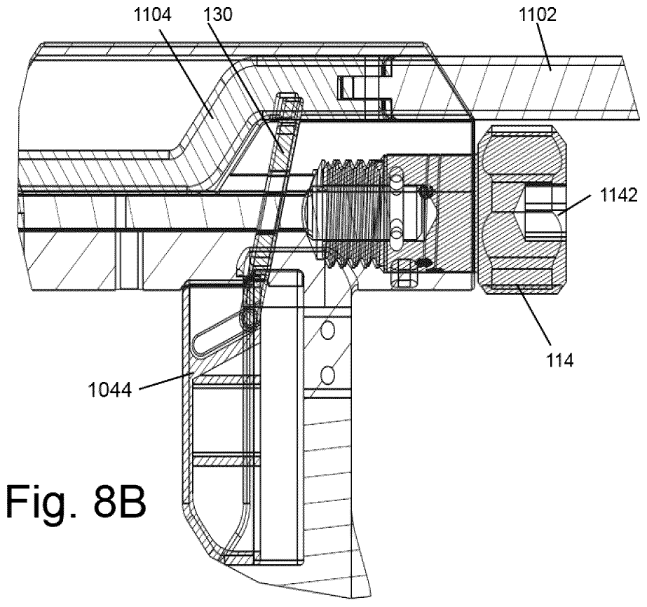

Once the distraction member 124 or 126 is positioned, a knob 114 is actuated so as to distract the intervertebral disc space and position the superior vertebral bone (i.e., the bone immediately above the target disc space) at a desired distance from and position relative to the inferior vertebral bone (i.e., the bone immediately below the target disc space). Specifically, in the embodiment shown in FIGS. 7A and 8B, the knob member 114 is disposed at a posterior surface of the body 105 of the implant placement instrument 100, and is configured to actuate the distraction member 124 or 126. For example, rotation of the knob member 114 in a first direction produces movement of the upper and lower plates away from one another towards the open (distracted) configuration (such as e.g., one of the configurations shown in FIGS. 9B, 9C, 9E or 11B), whereas rotation in an opposite direction causes movement of the upper and lower plates towards one another and into the closed (non-distracted) configuration (such as e.g., one of the configurations shown in FIGS. 9A, 9D, 9F or 11A). Alternatively, one of the plates may be movable and can be actuated via the knob, while the opposing plate is stationary. The knob 114 can be manually rotated by grasping the outer surface of the knob, or can be rotated by an instrument, such as, for example, a screw driver, a wrench, or the like that couples to a hex recess 1142 of the knob 114. In one exemplary implementation, a member 1102 attached at a posterior portion of the body 105 includes an aperture 11022 at a distal end portion 11024 which allows a screw driver to be advanced therethrough and into the recess 1142 of the knob 114 along trajectory D (see FIG. 7A).

After actuation of the knob 114 and distraction of the distraction member 124 or 126, an aperture 118 of the body 105 allows the operator to read a distance between a top surface of the upper plate 1242 and a bottom surface of the lower plate 1244. The reading can be used to select an appropriately sized implant to be advanced along the implant track 120 and into the distracted disc space, as well be discussed further below. The reading may be provided in a standard unit of measurement (such as millimeters, inches, etc.) or in selected letters and/or symbols that correspond to specific implant shapes and/or sizes. In the latter example, the implants usable with the implant placement instrument may be labeled with the corresponding letters and/or symbols.

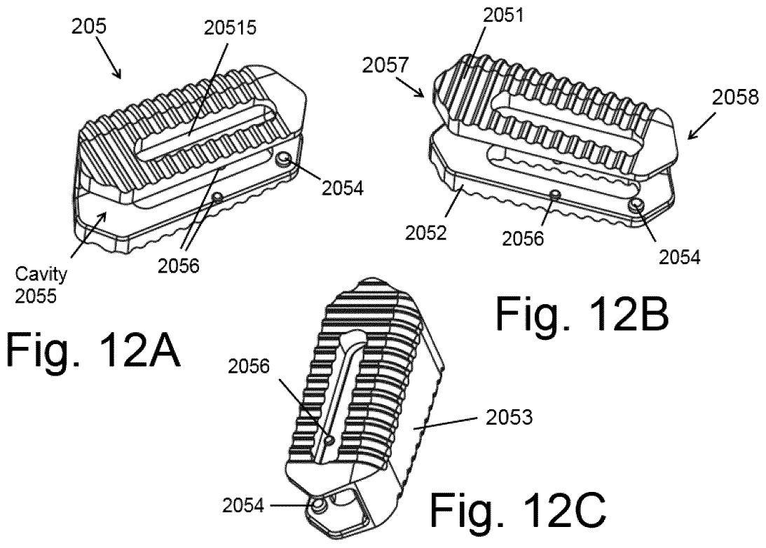

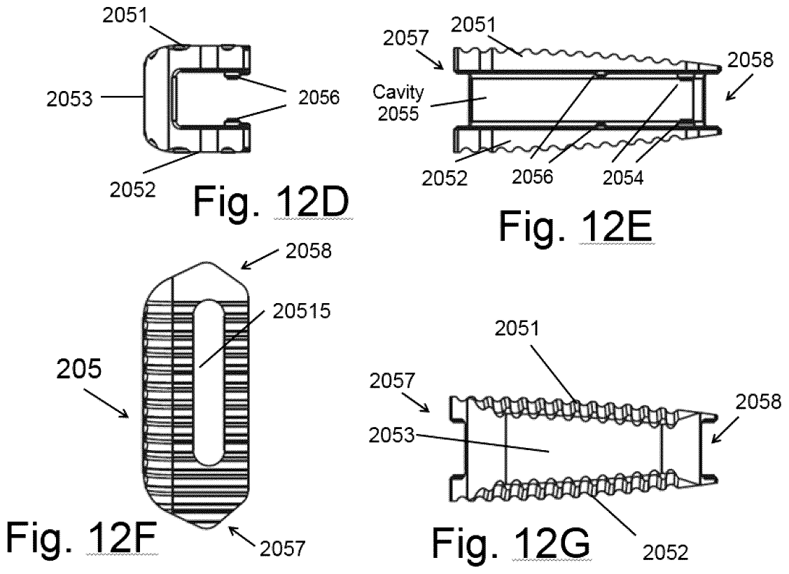

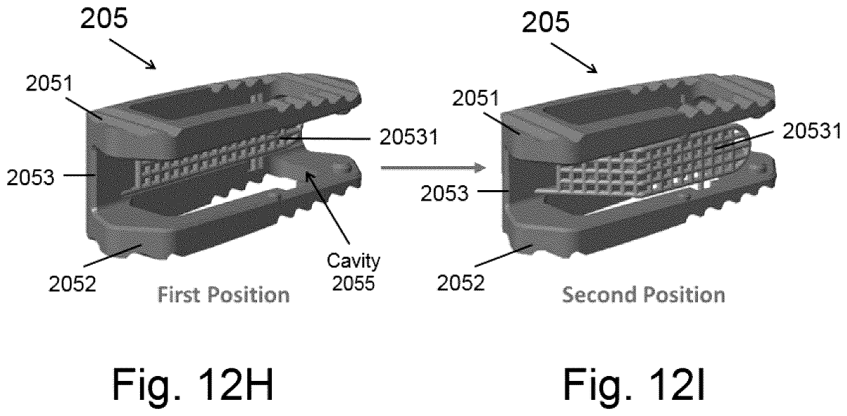

One exemplary embodiment of an implant 205 configured for use with the implant placement instrument 100 is shown in FIGS. 12A-12G. As illustrated therein, the implant 205 includes a superior member 2051 and an inferior member 2052. An outer surface of the superior member 2051 is configured to abut an inferior surface of the vertebral bone immediately superior to the target disc space in which the implant 205 is positioned, while an outer surface of the inferior member 2052 is configured to abut a superior surface of the vertebral bone immediately inferior to the target disc space. The superior and inferior members 2051 and 2052 are connected by a side member (or side wall) 2053. The superior, inferior, and side members define an internal cavity 2055, which is disposed within the implant 205 and sized to receive at least a segment of the implant track 120 therein.

Although the implant 205 is depicted as having only one side member, it will be appreciated that in alternate embodiments, the implant may include one or more additional side members or partial side members. For example, the implant 205 can optionally include a movable side wall 20531, as depicted in FIGS. 12H and 12I. Specifically, the moveable side wall 20531 is moveable between a collapsed configuration (FIG. 12H) where the movable side wall 20531 is substantially flush with the side wall 2053, and an extended configuration (FIG. 121) where the moveable side wall is disposed on an opposing lateral side of the implant 205 relative to the side wall 2053. The movable side wall 20531 may be comprised of a substantially elastic material. In one exemplary method of use, when loaded onto the implant track 120 the implant placement instrument 100, the movable side wall 20531 is compressed against the side member 2053 (in the collapsed configuration). When the insertion instrument 100 is removed from the disc space (after advancement of the implant into the disc space), the side wall is biased to the extended position. Stops (projections) on the top and bottom of the movable side wall may prevent the side wall from moving beyond the boundary of the implant (i.e., the projections are retained in the apertures in the superior and inferior members). It will be appreciated that the foregoing moveable side wall may enable (i) receipt of the exterior aspect of the implant track within the cavity and movement of the implant along with implant track in the collapsed configuration, (ii) added stability to the implant in the released configuration, and/or (iii) additional containment of bone graft material within the cavity.

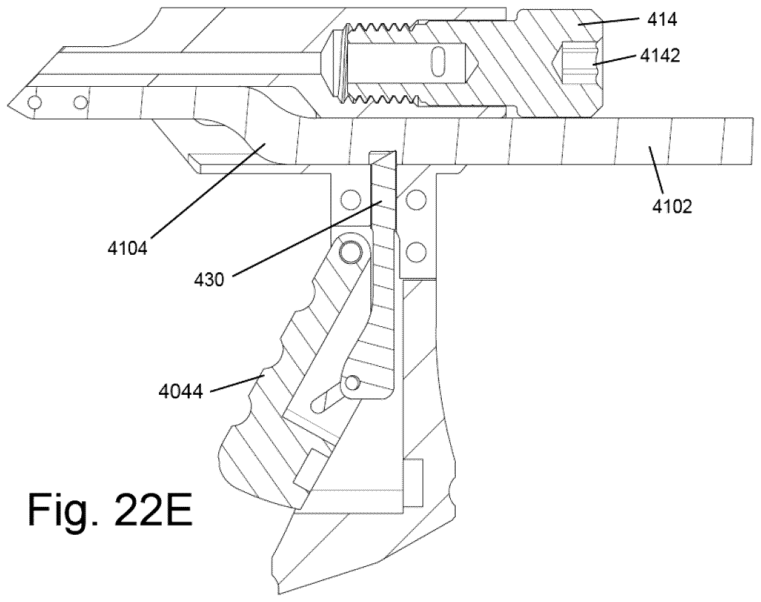

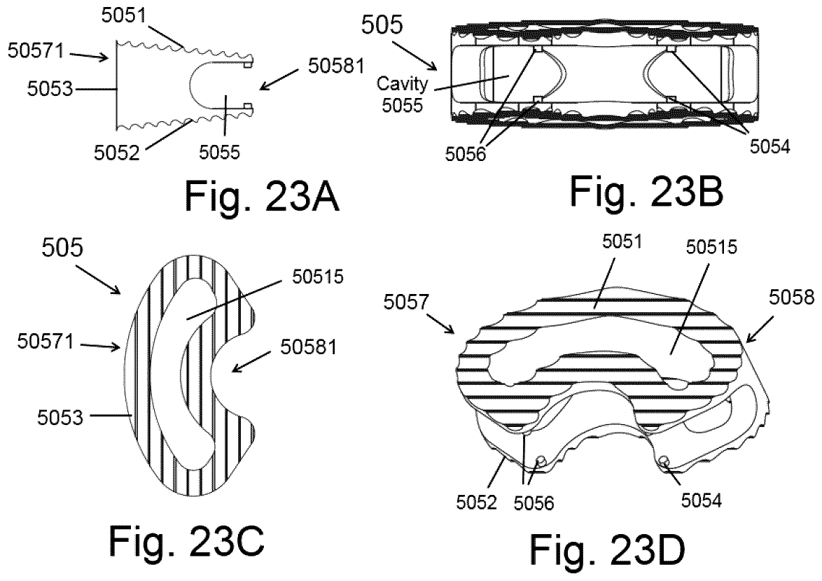

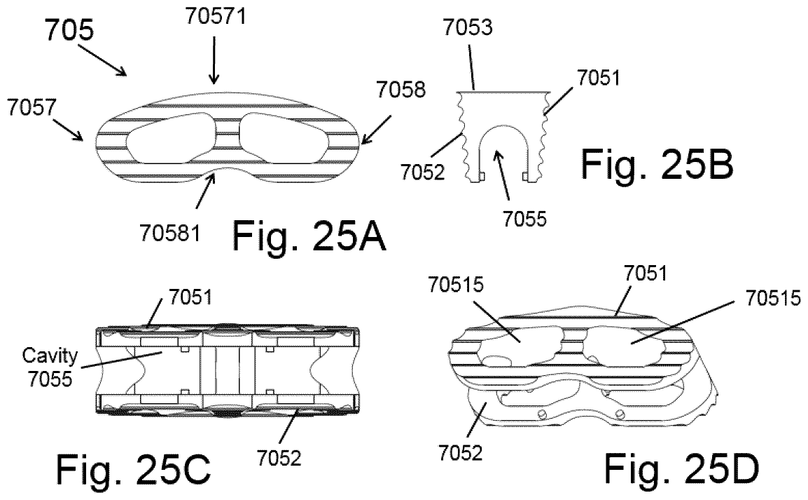

As best illustrated in FIGS. 12D and 12F, each of the superior and inferior members 2051 and 2052 includes substantially linear lateral edges and a substantially pointed or angled edge at each of a distal end 2057 and a proximal end 2058 of the implant 205. In alternate embodiments, the superior and inferior members can include one or more curved surfaces (see e.g., implants 505 and 705 depicted in FIGS. 23A-23D and 25A-25D, respectively), and/or one or more of the distal and proximal ends can have a different configuration (e.g., curved, blunted, etc.).

Turning again to FIG. 12E, each of the superior and inferior members 2051 and 2052 has an inclined exterior surface. Specifically, at the distal end 2057, each of the superior and inferior members 2051 and 2052 has a greater height than at the proximal end 2058 thereof. The foregoing features give the implant 205 a generally rectangular cuboid and tapered shape. In alternate embodiments, the superior and inferior members can have an equal height at each of the distal and proximal ends (giving the implant a non-tapered shape), or one or more of the superior or inferior members can be inclined to a greater degree than the exemplary embodiment shown in FIG. 12E.

As discussed elsewhere herein, it is contemplated a variety of implants of different shapes and/or sizes may be manufactured or provided to be usable with the implant placement instrument, and the appropriately shaped/sized implant can be selected from a pre-fabricated set of implants (or manufactured) to be specific to the condition of the target FSU in order to correct curvature or otherwise stabilize the FSU. In one embodiment, the implant 205 may be manufactured in various sizes and provided with the implant placement instrument as a kit. For example, the kit can include two lordotic options, 0° (parallel) and 8° (lordotic), with heights ranging from 8 mm to 16 mm and from 10 mm to 16 mm, respectively. Each of the implants has a width of approximately 10 mm, and can have a length of 25 mm to 30 mm.

As an alternative, the method of implant placement may include, prior to the surgical procedure, selecting a height to which the disc space will be distracted at the time of surgery (i.e., selecting the height as part of a pre-operative planning procedure performed at a date earlier than that of the surgery date). After selecting the height, the implant can be manufactured, such as e.g., via additive of subtractive 3-D printing, to the selected height, as well as dimensioned and contoured to conform to the patient-specific anatomy of the subject into which the implant will be subsequently implanted at surgery. The method may also include the use of computer-assisted navigation and/or robotics in the placement of the implant during the surgical procedure. Further, more than one target FSU may be treated/implanted at surgery and each treated FSU may be implanted with more than one implant.

Returning to FIGS. 12A-12G, each of the superior and inferior members 2051 and 2052 includes surface features (e.g., indentations and protrusions, endplate engaging surfaces, and/or anti-migration teeth) which increase implant fixation and anchor the implant onto adjacent bone. Although not shown, the side member 2053 can additionally include similar surface fixation features. Further, the surfaces of the members 2051, 2052 and/or 2053 may be coated or manufactured with an osteo-conductive bioactive material (such as, e.g., demineralized bone matrix, hydroxyapatite, and the like) and/or an osteo-inductive bioactive material (such as, e.g., Transforming Growth Factor “TGF-B,” Platelet-Derived Growth Factor “PDGF,” Bone-Morphogenic Protein “BMP,” and the like), which each promote bone formation. Furthermore, the surfaces of the implant 205 may be coated and/or manufactured with a textured or a porous ingrowth surface (such as, e.g., titanium wire mesh, plasma-sprayed titanium, tantalum, porous CoCr, and the like), provided with a bioactive coating (such as, e.g., a coating comprising tantalum, and/or helical rosette carbon nanotubes or other carbon nanotube-based coating) in order to promote bone in-growth and establish a mineralized connection between the bone and the implant.

Moreover, after implantation, the cavity 2055 may be filled with a bone forming material so as to enable a bony fusion that extends from the immediately superior vertebral bone, across the implant (via cavity 2055) and onto the immediately inferior vertebral bone. Each of surfaces 2051, 2052 and 2053 can include one or more apertures (such as, for example, an aperture 20515 within the superior surface 2051 shown in FIG. 12F) that allow direct communication between the cavity and the exterior of the implant. In this way, bone forming material placed into the cavity can form a bone bridge (i.e., fusion mass) across one or more of the the apertures and fuse the superior and inferior vertebral bones to one another. Each of the aforedescribed features can reduce the likelihood of implant dislodgement from the implantation site within the disc space.

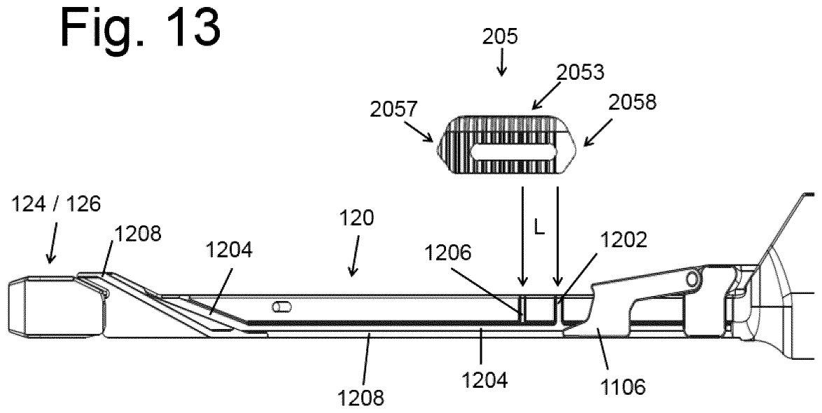

As can be seen in FIG. 13, once the distraction and/or positioning of the vertebral bones is performed and an appropriately sized/shaped implant has been selected, the implant 205 can be mounted onto an outer aspect of the implant track 120 for slidable delivery of the implant to the distracted disc space. Specifically, the implant 205 is mounted onto the implant track 120 along a direction L such that a pair of recesses 1202 and 1206 are aligned with and respectively receive complimentary protrusions of the implant. Accordingly, an upper surface of the implant track 120 engages with an interior surface of the superior member 2051, while a lower surface of the implant track 120 engages with an interior surface of the inferior member 2052.

As illustrated in 12A-12E and 12G, in one embodiment, a first pair of protrusions 2054 are disposed on an interior surface of each of the superior and inferior members 2051 and 2052 proximate to the proximal end 2058 of the implant 205, and are configured (sized and/or shaped) to be received within recesses 1202 and grooves 1204 (on opposing sides of the implant track 120) of the implant placement instrument 100. A second pair of protrusions 2056 are disposed on the interior surface of each of the superior and inferior members 2051 and 2052 at a central region of the implant 205, and are configured (sized and/or shaped) to be received within recesses 1206 and grooves 1208 (on opposing sides of the implant track 120) of the implant placement instrument 100. In one implementation, the protrusions have a head region that is greater in width/diameter than a stem region thereof (which may increase retainment of the implant to the implant track), or, in another implementation, the protrusions may be substantially cylindrical (which may increase ease of advancement of the implant along the implant track).

The protrusions are each (generally) disposed on the interior surfaces of the superior and inferior members on an opposing side of the implant 205 relative to the side member 2053. Accordingly, when the implant 205 is mounted onto the implant track 120, the side member 2053 is oriented away from the track. It is noted that, in the present embodiment, the protrusions 2054 are of different diameter, length/shape, and position within the cavity 2055 relative to the protrusions 2056, which thereby enables the first and second sets of protrusions to interact with and be slid within different (i.e., complimentarily configured) recesses and grooves when mounted onto and advanced down the implant track 120.

In alternate embodiments, the implant and the implant placement instrument can be configured with additional or fewer complimentary protrusions and grooves (such as e.g.,, having protrusions on an interior surface of only one of the superior and inferior members), and/or the protrusions can be of an identical or similar configuration (such as e.g., having two sets of protrusions of an equal diameter on each of the superior and inferior members). Further, in additional alternate embodiments, the interior surface of the side wall and an outer aspect of the implant track can be configured to have complimentary protrusions and grooves. Yet further, in additional alternate embodiments, the interior surfaces of the implant can include one or more grooves therein, which are configured to receive a rail or elongate projection on an exterior surface of the implant track. It will be appreciated that various combinations of the foregoing embodiments for engagement of the implant and the implant track are contemplated herein.

In order to enable the aforementioned mounting and sliding of the implant down the implant track, it is noted that the recesses 1202 are continuous with the grooves 1204, and the recesses 1206 are continuous with the grooves 1208 (on each side of the implant track 120). Thus, after the implant is mounted onto the implant track, a release member 1044 (FIG. 7A) on the handle 104 of the implant placement instrument 100 can be depressed to release an implant pusher 110. As shown in FIG. 7A, the implant pusher 110 includes the proximal member 1102, an intermediate member 1104, and a distal, rotatable member 1106. As can be seen in FIG. 8B, a bar 130 is coupled to the release member 1044 and is configured to enable actuation of the implant pusher 110 via the release member 1044. Specifically, when the release member 1044 is fully extended (i.e., when member 1044 is not depressed), one end of the bar 130 is positioned within a recess of the intermediate member 1104 so that the pusher 110 is immobilized relative to the body 105 and cannot be moved along the track 120. When the release member 1044 is depressed, the bar 130 is withdrawn from the recess within the intermediate member 1104 so that a force (such as, for example, a force exerted by a mallet, a manual force, a machine guided force, or a force from a spring loaded device) is applied to the proximal end 11024 will permit the (now-unconstrained) pusher 110 to advance along the implant track 120 and push the implant 205 towards the distraction member 124 or 126. Specifically, the implant 205 is engaged by the distal, rotatable member 1106 of the pusher 110 as it is advanced along the implant track 120. The implant 205 is retained onto the implant track 120 by the interaction of the protrusions 2054 and 2056 with the grooves 1204 and 1208 respectively. It is noted that the distal end 2057 is a leading edge or side of the implant, while the proximal end is a lagging edge or side of the implant when the implant 205 is advanced along the implant track 120.

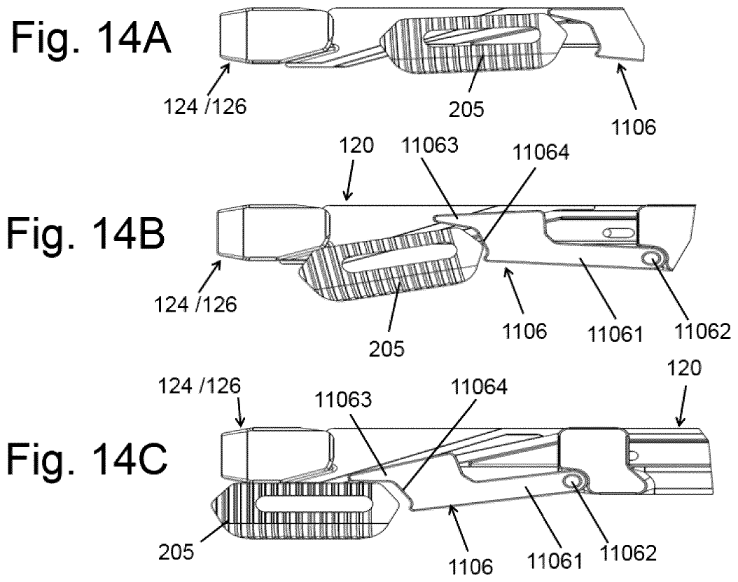

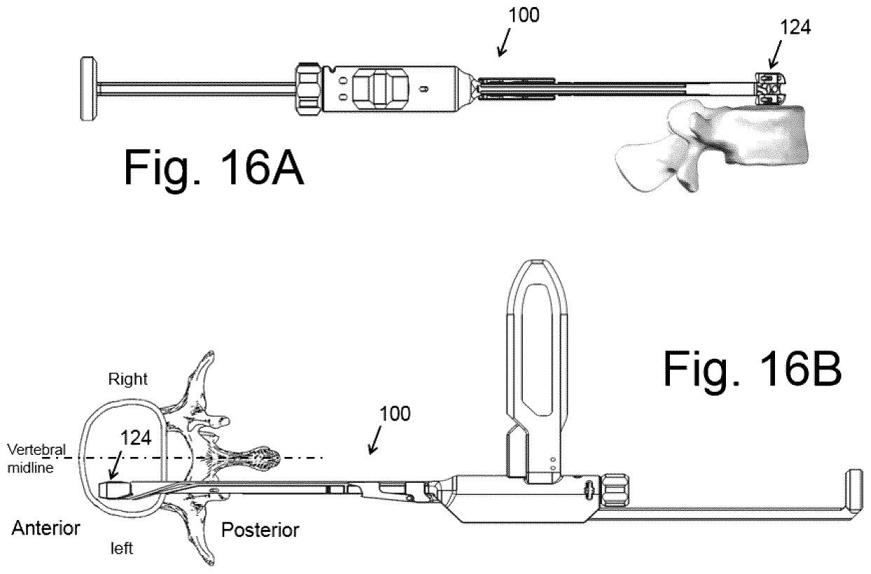

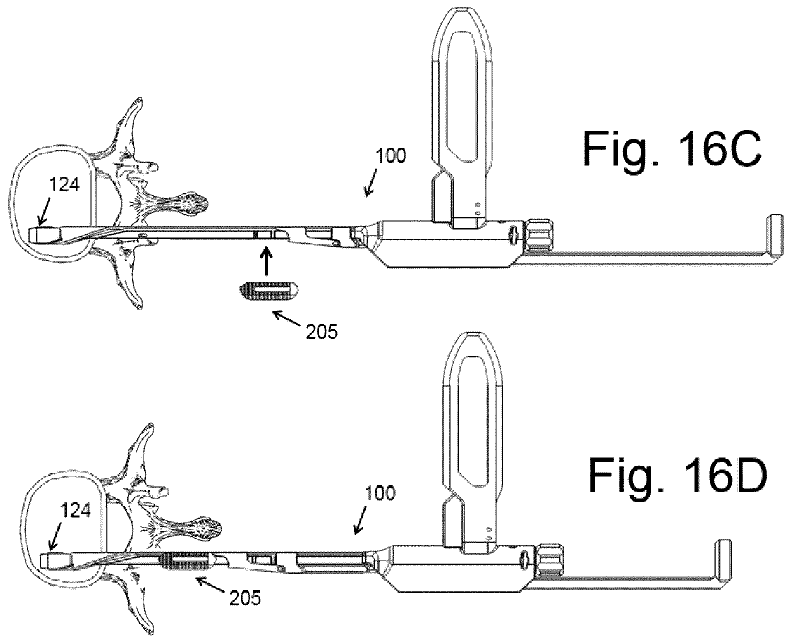

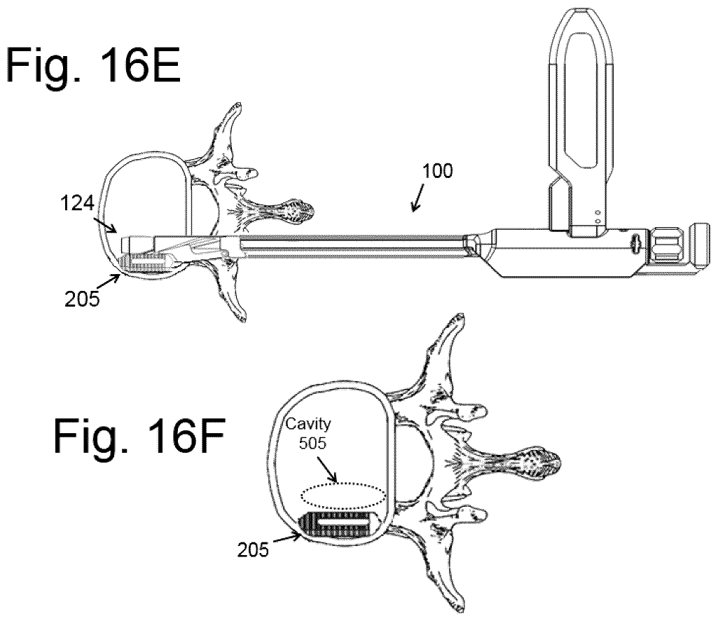

In the present embodiment, the implant is first advanced through a proximal region of the grooves along a linear pathway, and at the distal segment of the implant track the grooves include turns (curvatures or bends) so that the implant is ultimately positioned within the disc space at a lateral side of (and/or anterior to) the distraction member (as depicted in FIG. 16E and discussed below). FIGS. 14A-14C show step-wise movement of the implant 205 along the distal segment of the implant track 120. Specifically, the distal segment of the each of the grooves each 1204 and 1208 includes a bend, such that the terminal portion of each of the grooves is oblique or transverse relative to a longitudinal axis of the implant track 120. As the implant 205 is moved along terminal portion of the grooves 1204 and 1208 (via engagement with the distal, rotatable member 1106 of the implant pusher 110), the implant 205 moves laterally relative to the implant track 120 (i.e., onto a left side or a right side of the implant track), as well as forward toward the distal end of the implant track.

As can be seen in FIGS. 14A-14C, the distal, rotatable member 1106 of the implant pusher 110 includes (at a proximal end thereof) an arm 11061 rotatably connected to the intermediate member 1104 via a pin 11062, and (at a distal tend thereof) an implant engagement portion comprising a protrusion 11063 and a shelf 11064. The protrusion 11063 is configured to engage the proximal end of the implant 205 at a side surface thereof in order to guide the implant through the bend at the terminal portion of the grooves 1204 and 1208 (FIG. 14A). Subsequently, the shelf 11064 is configured to engage an extended portion of the proximal end of the implant 205, and catch the extended portion on a raised lip of the shelf 11064. Such engagement enables further lateral (and forward) advancement of the implant 205, as well as rotation of the arm 11061 about an axis of the pin 11062 (FIGS. 14B and 14C), until the implant 205 is guided through an end of the grooves 1204 and 1208 (where the protrusions 2054 and 2056 disengage from the grooves 1204 and 1208, respectively) and is positioned adjacent to the distraction member 124 or 126.

While the longitudinal axis of implant track is aligned with the longitudinal axis of the distraction member in the present embodiments (as shown in e.g., FIG. 13), the latter may be alternatively offset from the longitudinal axis of the implant track. When the longitudinal axes of distraction member the implant track are offset relative to one another, the implant placement instrument may be configured, for example, so as advance the implant in a substantially linear trajectory along the implant track and into the target disc space (rather than including a curved trajectory at the distal end of the implant track).

### Methods of Use

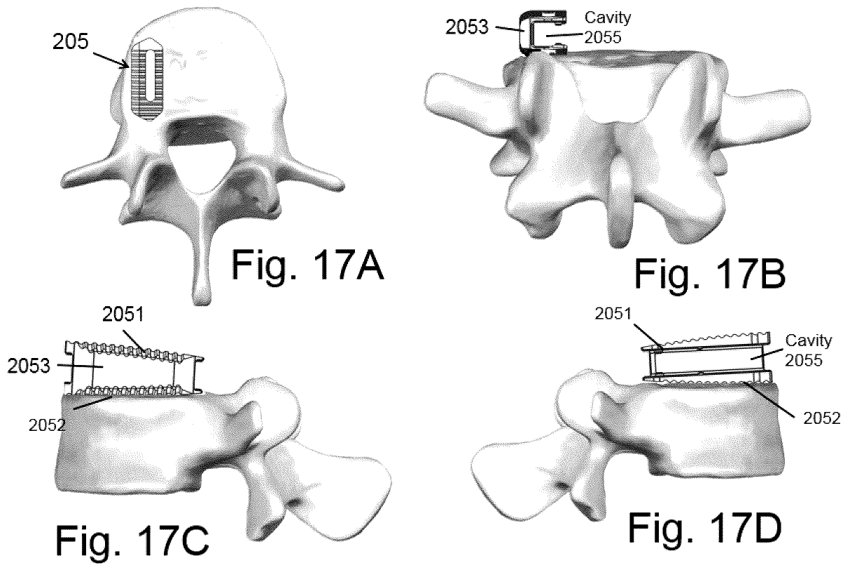

FIGS. 16A-17F illustrate exemplary methods for positioning a portion of the implant placement instrument within a target intervertebral disc space, mounting of the implant onto the implant track, advancement of the implant down the implant track and into the disc space, and removal of the implant placement instrument from the disc space.

Specifically, the disc space may be approached using any applicable corridor to the spine—such as, for example, via one or more of anterior, antero-lateral, lateral, postero-lateral and posterior tissue corridors. In one embodiment utilizing the postero-lateral and/or posterior approaches, for example, the paraspinal muscles are retracted after placement of a skin incision. A corridor is then developed to the postero-lateral and/or posterior aspect of the spinal column and at least a segment of the lamina and/or facet joint(s) can be removed. If necessary, a posterior aspect of the thecal sac can be exposed. The posterior aspect of the target disc space is then exposed through a corridor that is lateral to the thecal sac. The thecal sac is retracted gently in the medial direction, and the posterior aspect of the target disc space can be identified. The annulus fibrosis is incised and at least a segment of the native disc space is removed. After the preparation of the bony end plate, the target disc space is ready for advancement of an implant therein.

As discussed elsewhere herein, the implant placement instrument 100 may be hand held (using handle 104) during the procedure and/or the placement instrument may be anchored to a bony surface of the vertebral column. The placement instrument 100 may be additionally or alternatively attached to a coupler or segment of an articulating retention arm, which is anchored to the operating room table upon which the patient is positioned. Frame devices that anchor surgical instruments to the operating table are known in the art, and an exemplary articulating retention arm is show in FIG. 15. In the illustrated example, an articulated frame 905 has a member 9052 that reversibly attaches to the operating table onto which the patient is positioned. A member 9056 is adapted to reversibly and rigidly clamp onto (or otherwise couple with) a segment of the implant placement instrument 100 (such as, for example, a segment 1046 shown in FIG. 7A). A member 9054 is adapted to reversibly transition the frame 905 from a first state (i.e., a movable state where articulation of the frame segments is enabled) to a second state (i.e., a locked state where the frame segments are rigidly locked to one another). While the exemplary articulated frame 905 is illustrated, it is understood that other positioning or retaining devices may be alternatively (or additionally) used. Other exemplary positioning or retaining devices that can be used with the implant placement instruments described herein are disclosed in U.S. Pat. Nos. 4,254,763; 5,908,382; 6,302,843; 6,709,389; and 7,156,806, each of which is incorporated by reference herein in its entirety.

FIGS. 16A-16F illustrate a first exemplary embodiment of a method of use of the implant placement instrument 100. As depicted therein, the implant placement instrument 100 inserted into the target disc space via a posterior approach. As depicted in FIG. 16A (and discussed above with reference to FIGS. 7A-9F), after the distal end of the implant placement instrument is advanced into the target disc space, the knob 114 is actuated and the distraction member 124 is moved from the closed (non-distracted) configuration to the open (distracted) configuration in order to distract the disc space to a desired height and/or otherwise reposition the superior or inferior vertebral bones (such as, e.g., to correct anterior spondylolisthesis as shown in the exemplary distracted configuration of FIG. 9C, or to correct lateral spondylolisthesis as shown in the exemplary distracted configuration of FIG. 9E).

After the desired height of the disc space and position of the the superior or inferior vertebral bones is achieved, the implant size and/or shape may be selected based on the reading shown in the aperture 118. In alternate embodiments, the implant size and/or shape can be selected based on other criteria, such as, e.g., imaging of the target spinal segment.

Once the appropriate implant is selected (such as, e.g., implant 205 shown in FIGS. 12A-12G), it is mounted onto the implant track 120 as shown in FIG. 16C. The release member 1044 is then depressed and the implant pusher 110 is pushed forward (via, e.g., hammering with a mallet, manual movement, or machine guided movement, and/or a spring loaded device) such that the distal, rotatable member 1106 engages with the implant 205. The rotatable member 1106 advances the implant 205 along tract 120, as shown in FIG. 16D.

FIG. 16E illustrates delivery of the implant 205 into the disc space at a lateral position relative to the distraction member 124. In one embodiment of the method of implantation, the implant 205 is positioned to abut at least a portion of the inferior apophyseal ring of the superior vertebral bone and/or the superior apophyseal of the inferior vertebral bone. In an alternate embodiments, an implant can be positioned anterior to the distraction member or medial relative to the distraction member.

After the implant 205 is positioned within the target disc space, the knob 114 is actuated (e.g., actuated in a reverse direction) and the distraction member 124 is decreased in height to move the distraction member into the closed (non-distracted) configuration. The implant placement instrument 100 is then withdrawn from the disc space leaving the implant 205 positioned as shown in FIG. 16F.

FIGS. 17A-17D illustrate the implant 205 positioned within the disc space after the removal of the implant placement instrument 100 (the superior vertebral is not shown for diagrammatic simplicity).

In the embodiment of FIGS. 16A-16F, the distraction member 124 is a non-separable segment of the implant placement instrument 100 and is non-detachable therefrom. In other words, both of the distal end of the implant track and the distraction member are removed prior from the disc space prior to completion of the implantation procedure. Accordingly, after withdrawal of the placement instrument, a cavity 505 remains in the disc space at a location where the distraction member 124 had been positioned. Bone graft material may be packed into the cavity 2055 of the implant 205 and/or the cavity 505 (which is adjacent and medially disposed relative to the implant 205) so as to produce a bony fusion between the inferior surface of the superior vertebral bone and the superior vertebral surface of the inferior vertebral bone. It is noted that the bone graft material placed within the cavity 2055 may be in continuity with the bone graft placed within the cavity 505.

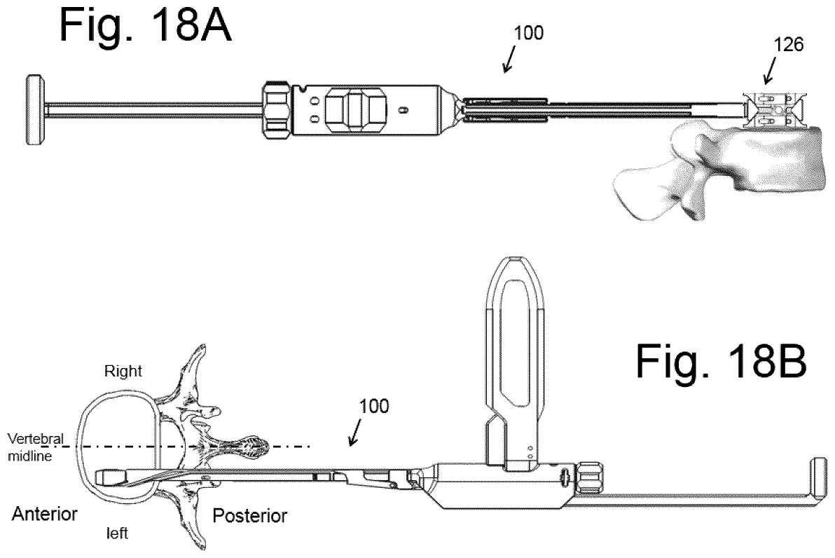

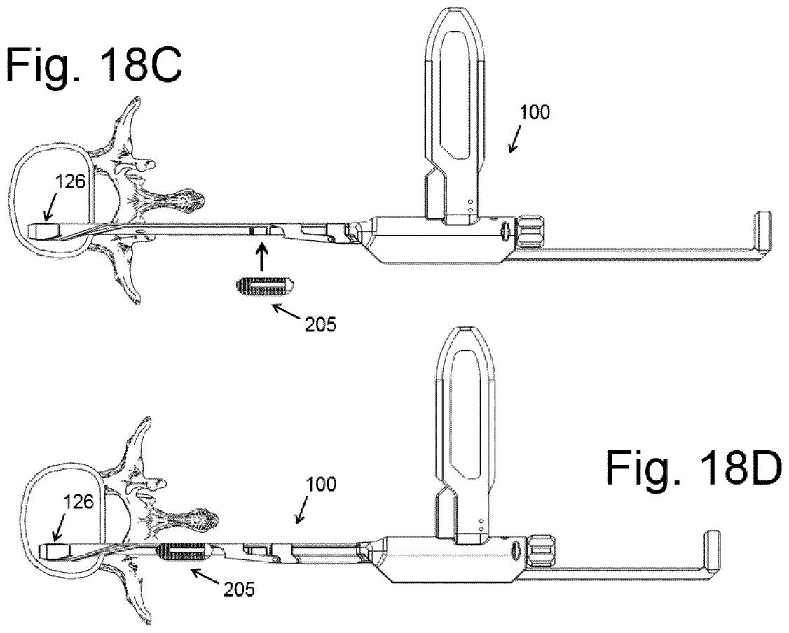

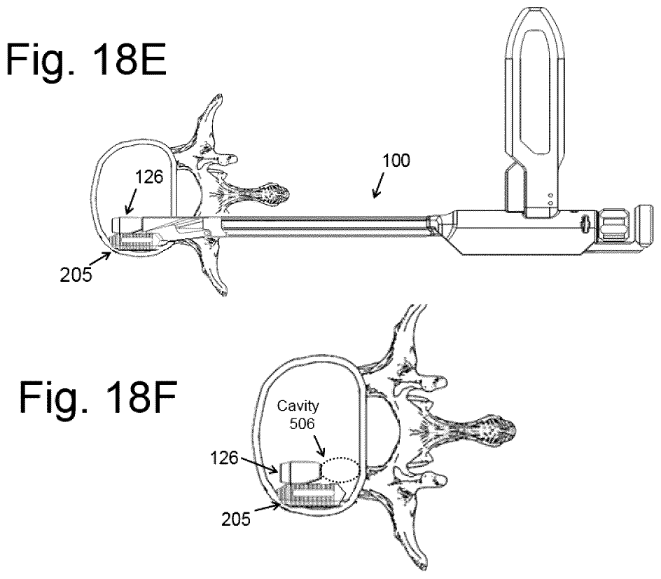

Turning now to FIGS. 18A-18F, another embodiment of a method of use of the devices disclosed herein is illustrated. First, the implant placement instrument 100 is inserted into the target disc space via a posterior approach. As depicted in FIG. 18A, after the distal end of the implant placement instrument is advanced into the target disc space, the knob 114 is actuated and the distraction member 126 is moved from the closed (non-distracted) configuration to the open (distracted) configuration in order to distract the disc space to a desired height and/or otherwise reposition the superior or inferior vertebral bones (such as, e.g., to correct lateral spondylolisthesis as shown in the exemplary distracted configuration of FIG. 11B). Similar to the method of use shown in FIGS. 16A-16F, after positioning of the implant placement instrument and distraction of the disc space (FIGS. 18A and 18B), the implant is selected (based on a size and/or configuration thereof) and mounted onto the implant track (FIG. 18C) and advanced along the track (FIG. 18D) and into the target disc space (FIG. 18E).

Different from the embodiment of FIGS. 16A-16F, in the present method, the distraction member 126 is a separate, detachable member that can be uncoupled from (reversibly attached to) the implant placement instrument 100 such that it remains in the disc space as an implant after the withdrawal of the implant placement instrument from the disc space. In other words, after the distraction member 126 is moved into the open (distracted) configuration and the implant 205 is positioned within the target disc space, the distraction member 126 is detached from the implant placement instrument 100 (via e.g., depression or actuation of a release member operatively coupled to the handle of the implant placement instrument 100) and the distraction member 126 retains its distracted height after the detachment. Specifically, once the desired distracted configuration is achieved, the distraction mechanism can be locked so as to make the configuration effectively permanent. Adhesives or yet other means for maintaining the desired position can be utilized as well.

The implant placement instrument 100 is then removed from the disc space leaving the implant 205 and the detached distraction member 126-as shown in FIG. 18F. Bone graft material may be packed into the cavity 2055 of the implant 205 and/or the cavity 505 (which is adjacent and medially disposed relative to the implant 205, and adjacent to and posterior of the detached distraction member 126) so as to produce a bony fusion between the inferior surface of the superior vertebral bone and the superior vertebral surface of the inferior vertebral bone.

While implantation of only one implant (i.e., the implant 205) is shown in FIGS. 16E-17D, and implantation of only one implant (i.e., the implant 205) and one detached distraction member (i.e., the distraction member 126) is shown in FIGS. 18E-18F, it is contemplated that additional implants (having the same or a different configuration as the implant 205) and/or additional detached distraction members (having the same or a different configuration from the distraction member 126) can be implanted into a single target disc space in order to disperse the load and/or alter the alignment across the FSU.

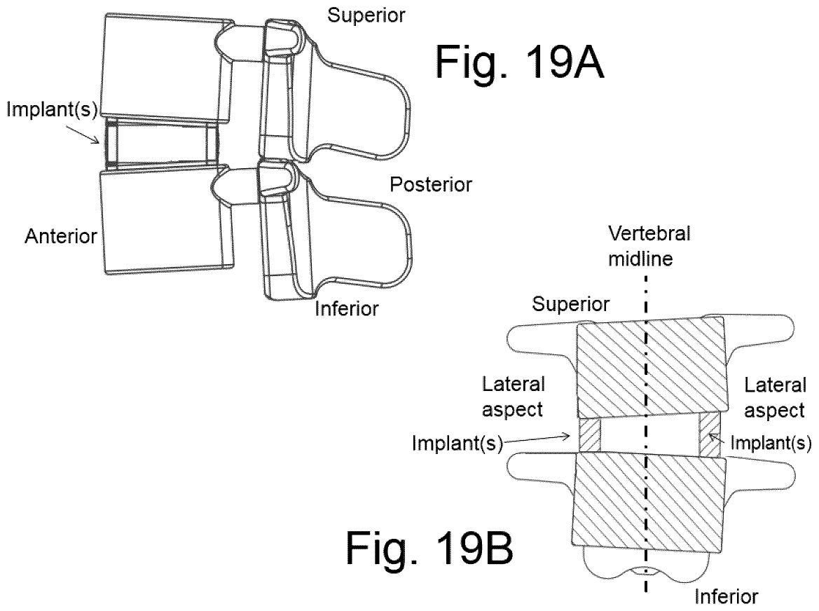

For example, FIGS. 19A and 19B illustrate how use of multiple implants (and/or detached distraction mechanisms of different shapes and/or sizes) can be used to alter the vertebral alignment within the vertebral column. Specifically, an implant (or detached distraction mechanism) may comprise a greater height at its anterior edge than at its posterior edge, as shown in FIG. 19A, so that a lordosis curve between the superior and inferior vertebral bones that abut the target disc space is altered (i.e., use of the implants or detached distraction mechanisms to a greater or lesser lordosis- or even leave the lordosis unchanged). Further, the implants (or detached distraction mechanisms) can be placed at opposing lateral sides of the target disc space so as to further alter the alignment of the implanted FSU. FIG. 19B illustrates that a first implant (or detached distraction mechanism) of a given height may be positioned at a first lateral side segment of the target disc space, and a second implant (or detached distraction mechanism) of a different height may be positioned at an opposing second lateral side segment of the target space, thereby altering the vertebral alignment in the coronal plane of the spinal column. In this way, this implant arrangement may be used in the treatment of spinal misalignment conditions such as, for example, scoliosis.

### Additional Spinal Stabilization Devices and Techniques

One or more supplemental fixation devices and/or methods may be utilized with the foregoing apparatus and methods in order to rigidly immobilize the superior and inferior vertebral bones of the target FSU (having the implant 204 and/or the distraction member 206 implanted therein). In one example, pedicle screw immobilization can be employed by the placement of one or more bone screw assemblies into the posterior aspect of the ipsilateral pedicle of each of the superior and inferior vertebral bones (e.g., a screw my enter each of the bones in proximity to position 811 shown in FIGS. 1A-1C).

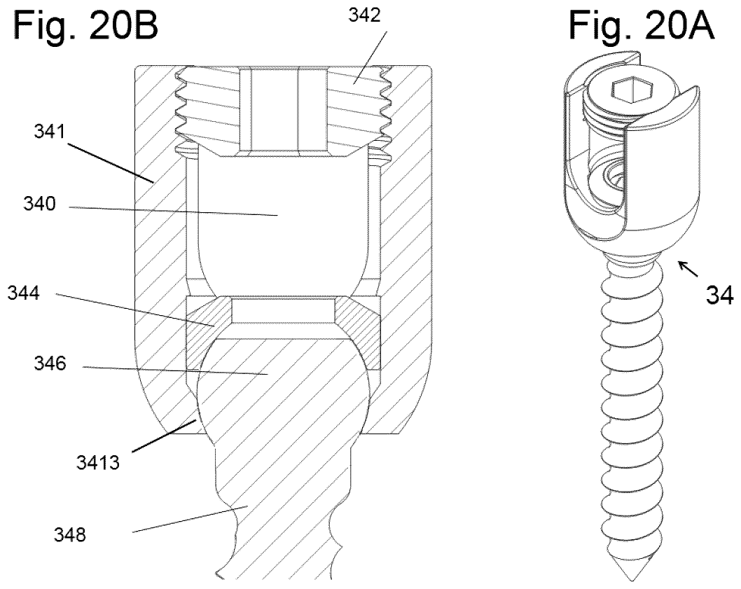

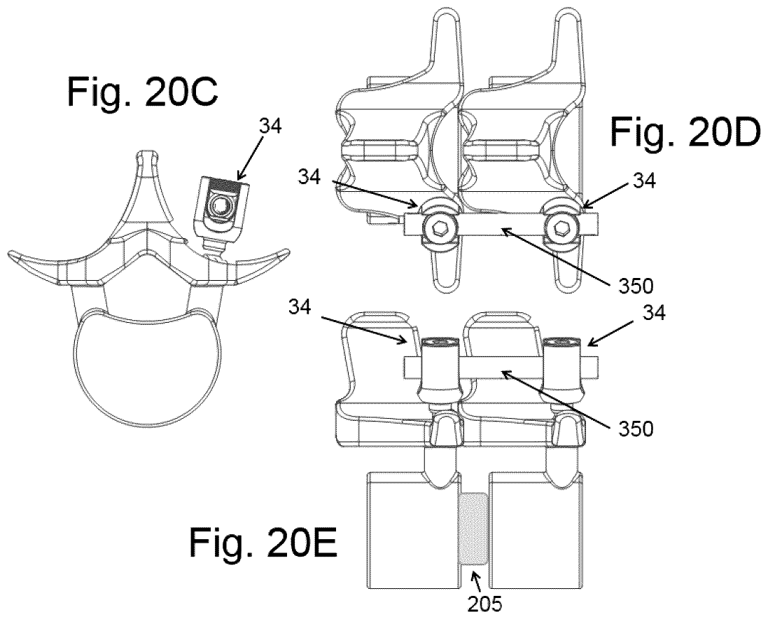

An exemplary pedicle screw assembly 34 is shown in FIGS. 20A and 20B. As depicted therein, the pedicle screw assembly 34 includes: a housing body 341 having a cavity 340 configured to receive an inter-connecting member (such as a rod), a set screw 342 that threadedly couples within a top portion of the cavity 340, a thrust washer 344 seated within a bottom portion of the cavity 340 (proximate to an anchor member-receiving portion 3413 of the housing body 341), and a bone anchor member including a head 346 and a threaded 348 configured for insertion into bone. In the assembly 34, the head 346 of the bone anchor member is seated within the anchor member-receiving portion 3413 and the threaded shank 348 of the bone anchor member extends out of an aperture that is positioned within the bottom of the anchor member-receiving portion 3413. It will be appreciated that the terms “above” and below” are relative and depend on the orientation of the assembly 34. As used herein, the assembly 34 is oriented with the set screw 342 at the superior aspect of the assembly and the threaded shank 348 at the inferior aspect of the assembly. Thus, the set screw is located “above” the bone anchor member and the cavity which receives the inter-connecting member receiving is disposed therebetween.

During use thereof (as shown in FIGS. 20C-20E), a connecting member 350 (such as a rod) is used to connect two or more of the assemblies 34, such as, e.g., connecting a first assembly 34 anchored into a superior vertebral bone and a second assembly 34 anchored into an inferior vertebral bone, the superior and inferior vertebral bones having one or more of the implants 205 (depicted in FIG. 20E) and/or the detached distraction members 126 implanted (not specifically depicted) therebetween. Each of the opposing ends of the connecting member 350 is received within the cavity 340 of one of the assemblies 34. Threaded engagement of the set screw 342 with the top portion of the cavity 340, the connecting member is movable within the cavity 340 relative to the housing body 341 and the head portion 346. Further, the head portion 346 can rotate within the anchor member-receiving portion 3413 such that a longitudinal axis of threaded shank 348 can assume different orientations and angles relative to a longitudinal axis of the housing body 341. With advancement of set screw 342, a compressive force is produced between the inter-connecting member 350 (contained within the inter-connecting member receiving portion 340) and the anchor member-receiving portion 3413 such that the interconnecting member 350, the head portion 346 and the housing 341 are immobilized relative to one another. In this way, the two assemblies are rigidly interconnected by the interconnecting member, and the procedure may optionally be repeated on the contra-lateral (opposing) side. Exemplary embodiments of pedicle screw fixation of adjacent vertebral bones (for use the the methods and apparatus described herein) are disclosed in U.S. Pat. No. RE37,665 and U.S. Patent Publication No. 2006/0084981, each of which is incorporated herein by reference in its entirety.

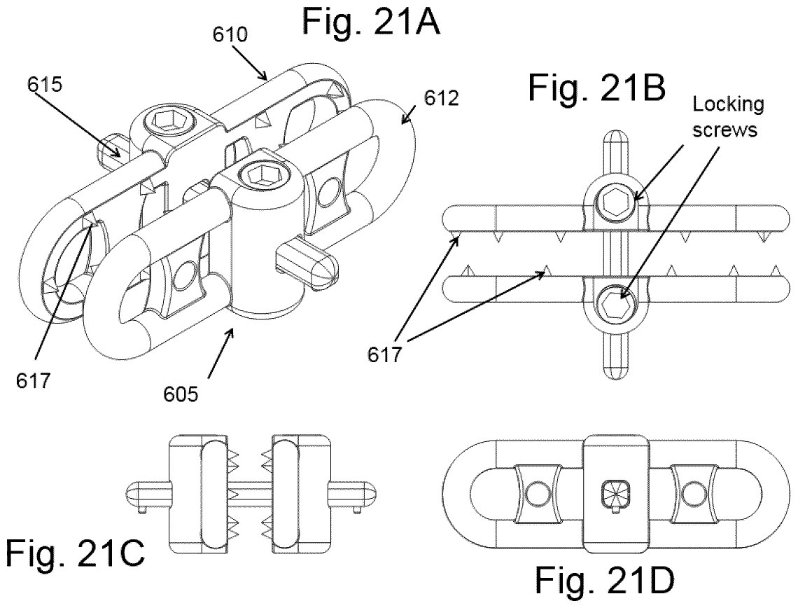

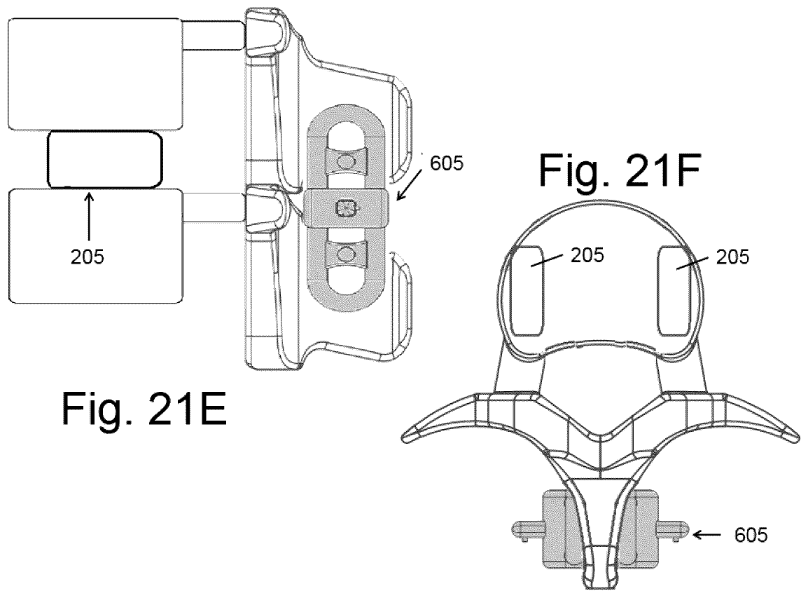

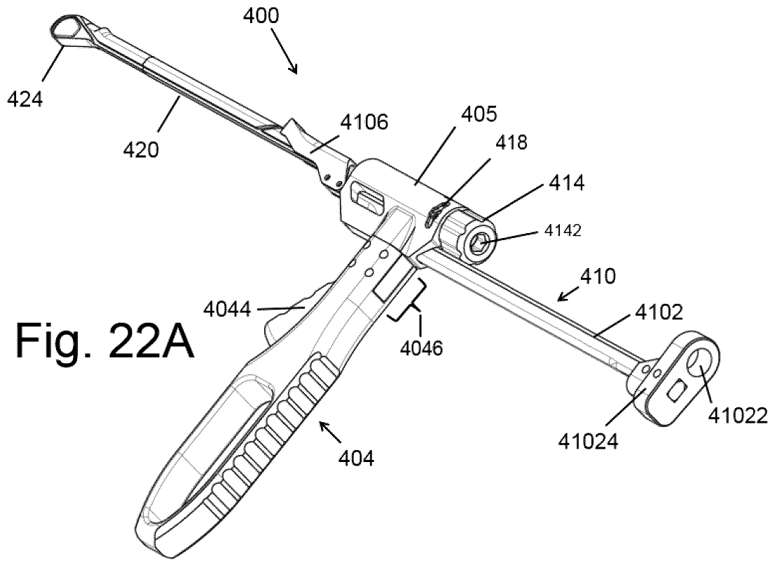

As an alternative (or in addition) to pedicle screw fixation, a spinous process fixation implant may be used for supplemental FSU stabilization. An exemplary spinous process fixation device 605 is illustrated in FIGS. 21A-21F. Specifically, the fixation device 605 includes a first member 610 and an opposing second member 612, which are configured to be adjoined via an interconnecting member 615 and attached onto opposing (contra-lateral) spinous processes of two adjacent vertebral bones. As depicted in FIGS. 21E and 21F, the spinous processes are forcibly captured between the first and second members 610 and 612. The interconnecting member 615 is then tightened or otherwise locked relative to the members 610 and 612, and prevents the members 610 and 612 from moving away from one another. A plurality of projections 617 on an interior surface of each of the members 610 and 612 penetrate the spinous processes and increase bone fixation onto the bone for the fixation device 605. In the present embodiment, two implants 205 interbody are implanted bilaterally within the target disc space. In the axial plane (FIG. 21F), the implants 205 provide two anterior column supports while the spinous process fixation device 605 provides posterior midline support. In the lateral view (FIG. 21E), the implants 205 for an anterior abutment surface and the spinous process fixation device 605 forms posterior abutment surface. Thus, the combined use of the implants and the spinous process fixation device forms a balanced three-point support of the vertebral bones. Additionally, bone graft material may be placed between the spinous process and/or lamina of the superior and inferior vertebral bones after appropriate decortication of the bone at the intended graft recipient site, and/or one or more detached distraction members may be implanted within the disc space.

### Alternate Configurations

It is further contemplated that, in alternate embodiments, the distraction member and/or the implant track could be configured to include a curvilinear segment. In such embodiments, an implant may follow a trajectory down the implant track that is at least partially curvilinear (and may have a segment of defined a specified curvature), or the implant trajectory may also (or instead) comprise a segment that at least partially follows a non-linear path around the distraction member.

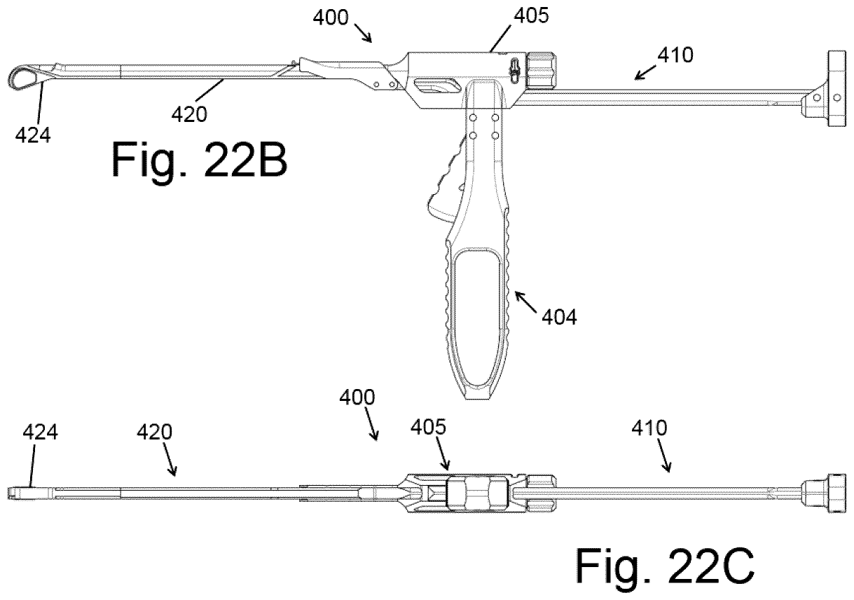

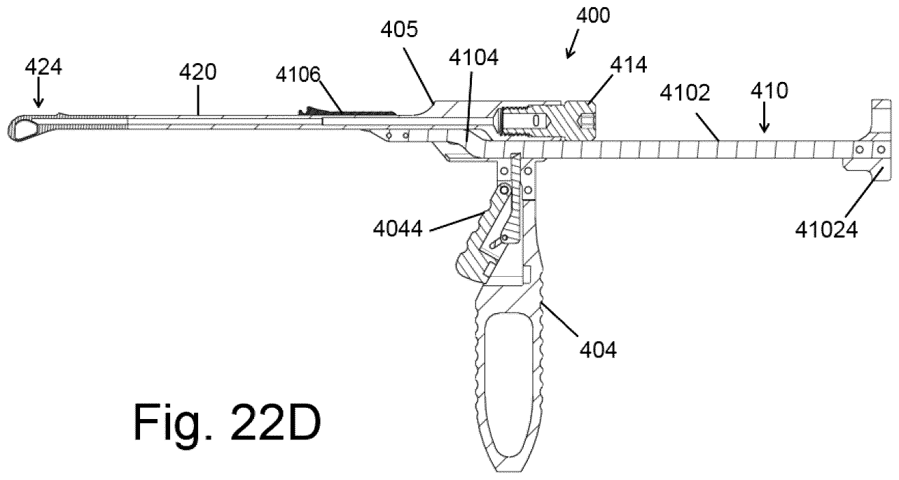

Other alternate embodiments are shown in FIGS. 22A-26D. As depicted therein, an implant placement instrument 400 includes a body 405 and a handle 404 extended outwardly therefrom. An implant track 420 extends outwardly from an anterior surface of the body 405, and a distraction member 424 extends outwardly from a distal end of the implant track 420.

Although not specifically shown, it will be appreciated that the distraction member 424 may have a similar configuration and method of operation/use to those discussed above with reference to distraction members 124 and 126. For example, the distraction member 424 can include upper and lower plates that are (reversibly) moveable from a (closed) non-distracted configuration to an open (distracted) configuration via actuation of a knob 414, which can be manually rotated by grasping the outer surface of the knob, or can be rotated by an instrument, such as, for example, a screw driver, a wrench, or the like that couples to a hex recess 4142 of the knob 414. In one exemplary implementation, a member 4102 attached at a posterior portion of the body 405 includes an aperture 41022 at a distal end portion 41024 configured to enable a screw driver to be advanced therethrough and into the recess 4142 of the knob 414. After movement into the distracted configuration, an aperture 418 of the body 405 allows the operator to read a distance between the upper and lower plates for selection of an appropriately sized implant to be advanced along the implant track 420 and into the distracted disc space. In one implementation, the distraction member 424 is non-detachable and is configured for removal from the disc space after delivery of the implant thereto. In another implementation, the distraction member 424 is detachable and is configured to be implanted within the disc space after delivery of the implant thereto.

A first exemplary embodiment of an implant 505 configured for use with the implant placement instrument 400 is shown in FIGS. 23A-23D. As illustrated therein, the implant 505 includes a superior member 5051 and an inferior member 5052. An outer surface of the superior member 5051 is configured to abut an inferior surface of the vertebral bone immediately superior to the target disc space in which the implant 505 is positioned, while an outer surface of the inferior member 5052 is configured to abut a superior surface of the vertebral bone immediately inferior to the target disc space. The superior and inferior members 5051 and 5052 are connected by a side member (or side wall) 5053. In alternate embodiments, one more additional or partial side walls may additionally be included in the implant (such as e.g., a flexible/movable side wall). As best illustrated in FIGS. 23A and 23B, the superior, inferior, and side members define an internal cavity 5055, which is disposed within the implant 505 and sized to receive at least a segment of the implant track 520 therein.

Different from the implant 205 show in FIGS. 12A-12G, each of the superior and inferior members 5051 and 5052 includes substantially continuously curved lateral edges and a substantially linear edge at each of a distal end 5057 and a proximal end 5058 of the implant 505. More specifically, a first lateral side 50571 of the implant 505 (at a closed side of the cavity 5055) has a convex curvature, while a second lateral side 50581 thereof (at an open side of the cavity 5055) has a concave curvature. As can be seen in FIG. 23A, each of the superior and inferior members 5051 and 5052 has an inclined exterior surface. Specifically, at the first lateral side 50571, each of the superior and inferior members 5051 and 5052 has a greater height than at the second lateral side 50581 thereof. The foregoing features give the implant 505 a generally curved cuboid shape with tapered exterior surfaces inclined from the concave side to the convex side thereof.

In alternate embodiments, the superior and inferior members can have an equal height at each of the distal and proximal ends (giving the implant a non-tapered shape), or one or more of the superior or inferior surfaces can be inclined to a greater degree than the exemplary embodiment shown in FIG. 23A. Further, in other alternate embodiments the superior and inferior members can curved to a greater or lesser degree. One such alternate embodiment, a second exemplary embodiment of an implant 705 configured for use with the implant placement instrument 400, is illustrated in FIGS. 25A-25D. As can be seen therein, the implant 705 has a similar shape to implant 505, however, each of the superior and inferior members 7051 and 7052 includes a convex curvature a first lateral side 70571 of the implant 705 (at a closed side of a cavity 7055 which is closed by a side wall 7053) and a concave curvature a second lateral side 70581 thereof (at an open side of the cavity 5055), which each have a lesser degree of curvature as compared to the corresponding structures of the implant 505. Further, at each of a distal end 7057 and a proximal end 7058 of the implant 705, the implant includes curved edge (rather than a linear edge as in the implant 505).

As discussed elsewhere herein, it is contemplated a variety of implants of different shapes and/or sizes may be manufactured or provided to be usable with the implant placement instrument, and the appropriately shaped/sized implant can be selected from a pre-fabricated set of implants to be specific to the condition of the target FSU in order to correct curvature or otherwise stabilize the FSU. Alternatively, a pre-operative planning procedure may be carried out to select dimensions and/or configurations for one or more implants specific to the anatomy of the patient, and the one or more implants can be manufactured (such as e.g., via subtractive or additive 3-D printing) for later implantation thereof.

In the present embodiment, each of the superior and inferior members 5051 and 5052 of the implant 505 and the superior and inferior members 7051 and 7052 of the implant 705 includes surface features (indentations and protrusions) which increase implant fixation and anchor the implant onto adjacent bone. Although not shown, the side members 5053 and 7053 can additionally include similar surface fixation features. Further, the surfaces of the superior, inferior, and/or side members may be coated or manufactured with an osteo-conductive bioactive material (such as, e.g., demineralized bone matrix, hydroxyapatite, and the like) and/or an osteo-inductive bioactive material (such as, e.g., Transforming Growth Factor “TGF-B,” Platelet-Derived Growth Factor “PDGF,” Bone-Morphogenic Protein “BMP,” and the like), which each promote bone formation. Still further, the surfaces of the implants may be coated and/or manufactured with a textured or a porous ingrowth surface (such as, e.g., titanium wire mesh, plasma-sprayed titanium, tantalum, porous CoCr, and the like), provided with a bioactive coating (such as, e.g., a coating comprising tantalum, and/or helical rosette carbon nanotubes or other carbon nanotube-based coating) in order to promote bone in-growth and establish a mineralized connection between the bone and the implant.

Moreover, after implantation, the cavity 5055 and 7055 may be filled with a bone forming material so as to enable a bony fusion that extends from the immediately superior vertebral bone, across the implant (via cavity 2055) and onto the immediately inferior vertebral bone. Each of the superior, inferior and/or side members can include one or more apertures (such as, for example, an aperture 50515 of the implant 505 and apertures 70515 of the implant 705 shown in e.g., FIGS. 23C and 25D, respectively) that allow direct communication between the cavity and the exterior of the implant. In this way, bone forming material placed into the cavity can form a bone bridge (i.e., fusion mass) across one or more of the the apertures and fuse the superior and inferior vertebral bones to one another. Each of the aforedescribed features can reduce the likelihood of implant dislodgement from the implantation site within the disc space.

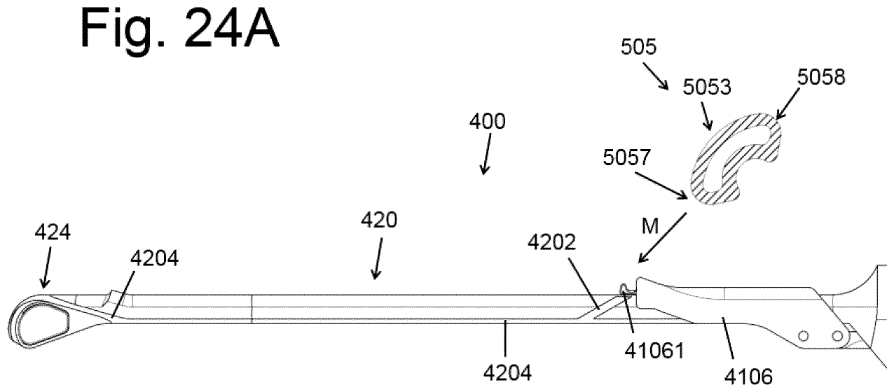

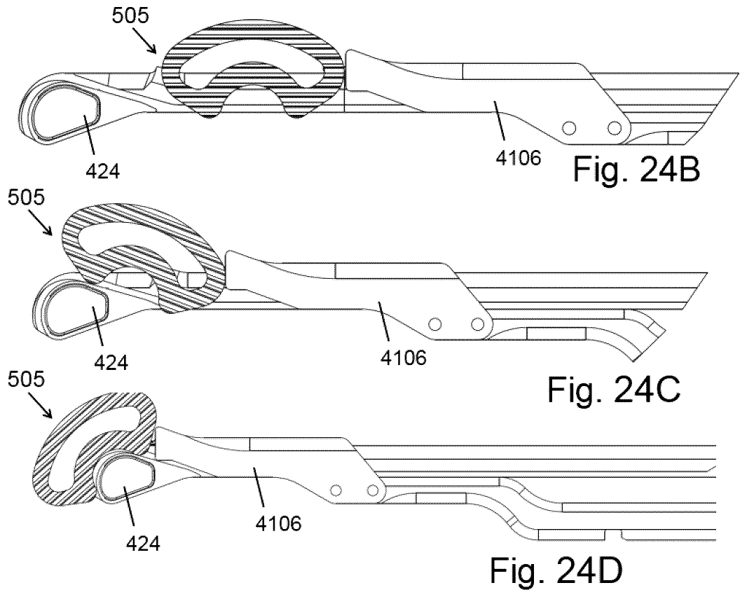

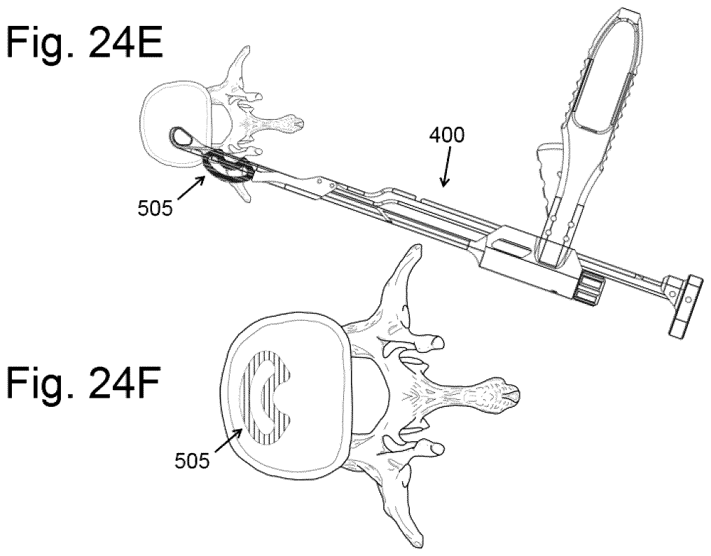

Turning to FIG. 24A, once the distraction and/or positioning of the vertebral bones is performed and an appropriately sized/shaped implant has been selected, the implant 505 can be mounted onto an outer aspect of the implant track 420 for slidable delivery to the distracted disc space. Specifically, the implant 505 can be mounted onto the implant track 420 along a direction M such that a recess 4202 is aligned with receives complimentary protrusions on the interior surfaces of the implant. Specifically, as illustrated in FIGS. 23A, 23B and 23D, a first pair of protrusions 5054 are disposed on an interior surface of each of the superior and inferior members 2051 and 2052 proximate to the proximal end 2058 of the implant 505, and a second pair of protrusions 5056 are disposed on an interior surface of each of the superior and inferior members 5051 and 5052 proximate to the proximal end 5058 of the implant 505. The protrusions 5054 and 5056 are configured (sized and/or shaped) to be received within recesses 4202 (and grooves 4204 (on opposing sides of the implant track 120).

Each of the protrusions 5054 and 5056 is disposed on an opposing lateral side of the implant relative to the side wall 5053 (i.e., at the second lateral side 50581). Accordingly, when the implant 505 is mounted onto the implant track 420, the side member 5053 is oriented away from the track. It is noted that, in the present embodiment, the protrusions 5054 are of a similar diameter and length/shape to the protrusions 5056. In alternate embodiments, the implant and the implant placement instrument can be configured with additional or fewer complimentary protrusions and grooves, and/or the protrusions can be of different sizes, lengths, shapes or other configurations. Further, the implant placement instrument can include an offset, additional grooves configured to receive one of the pairs of protrusions (similar to the configuration shown in the implant placement instrument 100).

In order to enable the aforementioned mounting and sliding of the implant down the implant track, it is noted that the recesses 4202 are continuous with the grooves 4204 (on each side of the implant track 420). Thus, after the implant is mounted onto the implant track, a release member 4044 (FIG. 22A) on the handle 404 of the implant placement instrument 400 can be depressed to release an implant pusher 410. As shown in FIGS. 22A and 22D, the implant pusher 410 includes the proximal member 4102, an intermediate member 4104, and a distal member 4106. As can be seen in FIGS. 22D and 22E, a bar 430 is coupled to the release member 4044 and is configured to enable actuation of the implant pusher 410 via the release member 4044. Specifically, when the release member 4044 is fully extended (i.e., when member 1044 is not depressed), one end of the bar 430 is positioned within a recess of the intermediate member 4104 so that the pusher 410 is immobilized relative to the body 405 and cannot be moved along the implant track 420. When the release member 4044 is depressed, the bar 430 is withdrawn from the recess within the intermediate member 4104 so that a force (such as, for example, a force exerted by a mallet, a manual force, a machine guided force, or a force from a spring loaded device) is applied to the proximal end 41024 will permit the (now-unconstrained) pusher 410 to advance along the implant track 420 and push the implant 505 towards the distraction member 424. Specifically, the implant 505 is engaged by the distal member 4106 of the pusher 410 as it is advanced along the implant track 420. The implant 505 is retained onto the implant track 420 by the interaction of the protrusions 5054 and 5056 with the grooves 4204.

In the present embodiment, the implant is first advanced through a proximal region of the grooves along a substantially linear pathway (which can be coincident with or parallel to a longitudinal axis of the implant track), and at the distal segment of the implant track the grooves include turns (curvatures or bends) so that the implant is ultimately positioned within the disc space with the distal end 5057 (i.e., a leading edge of the implant) anterior of the distraction member 424. FIGS. 24B-24C show step-wise movement of the implant 505 along the distal segment of the implant track 420. Specifically, the distal segment of the each of the grooves each 1204 and 1208 includes a bend, such that the terminal portion of each of the grooves is oblique relative to a longitudinal axis of the implant track 420. As the implant 505 is moved along terminal portion of the grooves 4204 (via engagement with the distal member 4106 of the implant pusher 410), the implant 505 moves laterally relative to the implant track 420 (as well as forward toward the distal end of the implant track).

As can be seen in FIGS. 24A-24D, the distal member 4106 of the implant pusher 410 includes (at a proximal end thereof) includes a blunted end that engages with the proximal end 5058 of the implant 505. The distal member 4106 further comprises a hooked protrusion 41061 (FIG. 24A) that is configured to reversibly latch or otherwise reversibly couple with the proximal end 5058 of the implant 505. The engagement between the hooked protrusion and the implant enables an operator or surgeon performing the procedure to optionally back the implant out (reverse movement along the implant track) if necessary. (Although not specifically shown, a similar hooked protrusion can be incorporated into the implant placement instrument 100 to enable reverse movement of the implant.)

During forward movement of the implant 505 along the distal segment of the implant track 420, the engagement of the blunted end of the distal member 4106 with the implant 505 enables concurrent forward and lateral advancement of the implant 505. Specifically, the concave curvature of the second lateral side 50581 is guided around a curved exterior surface of the distraction member 424, until the implant 505 is pushed through an end of the grooves 4204 (where the grooves 4204 disengage from the protrusions 5054) and the implant is positioned adjacent anterior to and lateral of the distraction member 424. It will be appreciated that the foregoing description can be similarly applied to use of the implant 705 with the implant placement instrument 400.

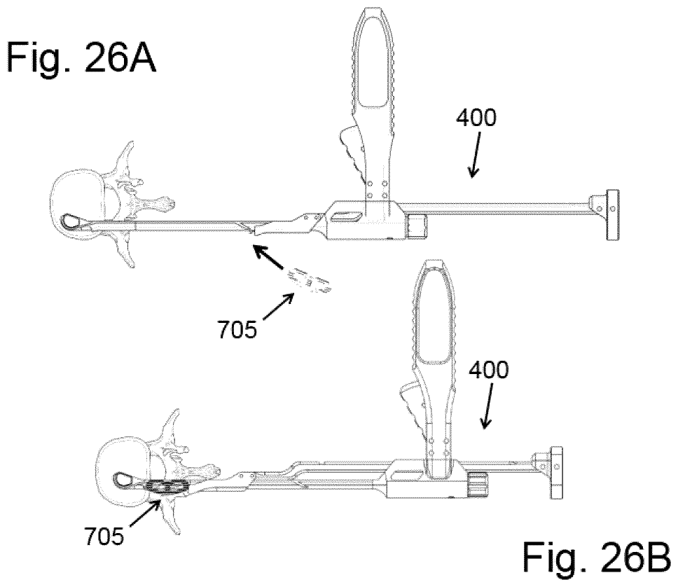

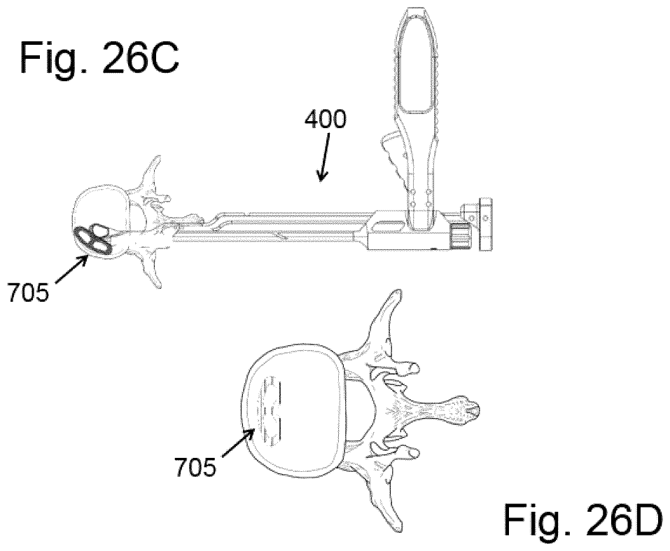

FIGS. 24E-24F and 26A-26D illustrate additional exemplary embodiments of methods of use of the devices disclosed herein. Specifically, FIGS. 24E and 24F depict the implant placement instrument 400 inserted into the target disc space via a postero-lateral approach for delivery of the implant 505 (having a greater degree of curvature than the implant 705). The postero-lateral approach enables sufficient space within the target FSU for the insertion of the implant 505 into the disc space and guidance around the curved exterior surface of the distraction member. After release from the implant placement instrument, the implant 505 is positioned proximate to the anterior portion of the disc space.

As depicted in FIGS. 26A-26D, the implant placement instrument 400 is inserted into the target disc space via a posterior approach for delivery of the implant 705 (having a lesser degree of curvature than the implant 505). The posterior approach enables sufficient space within the target FSU for the insertion of the implant 705 into the disc space and guidance around the curved exterior surface of the distraction member. After release from the implant placement instrument, the implant 705 is positioned proximate to the anterior portion of the disc space. In alternate embodiments, the implant placement instrument may be inserted along a posetero-lateral approach for delivery of the implant 705 to the disc space, and/or the implant can be positioned laterally, medially, or at another desired position within the disc space.

It will be appreciated that the methods of use discussed in detail above with reference to FIGS. 16A-18F are applicable to methods of using the implant placement instrument 400 with either of the implants 505 or 705.

It will be further appreciated that the disclosed device embodiments or any of their components can be made of any biologically adaptable or compatible materials. Materials considered acceptable for biological implantation are well known and include, but are not limited to, stainless steel, titanium, tantalum, combination metallic alloys, various plastics (such as PEEK and the like), resins, ceramics, biologically absorbable materials and the like. The system or any of its components can alternatively or additionally be entirely or partially made of a shape memory material or other deformable material. Any components may be also coated/made with osteo-conductive (such as demineralized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation.

Further, any surface may be made with a porous ingrowth surface (such as, for example, porous titanium, titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. The system or any of its components may be made by additive or subtractive manufacturing, such as, for example, 3D-printing.

While this specification contains certain specific features and attributes, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

It will also be recognized that while certain aspects of the disclosure are described in terms of a specific sequence of steps of a method, these descriptions are only illustrative of the broader methods of the disclosure, and may be modified as required by the particular application. Certain steps may be rendered unnecessary or optional under certain circumstances. Additionally, certain steps or functionality may be added to the disclosed embodiments, or the order of performance of two or more steps permuted. All such variations are considered to be encompassed within the disclosure disclosed and claimed herein.

1.-20. (canceled)

21. A device assembly for treatment of a functional spinal unit of a subject, the functional spinal unit comprising a superior vertebral bone, an inferior vertebral bone, and an intervertebral disc space positioned there between, the device assembly comprising:
a first implant sized to be at least partially received within the intervertebral disc space and comprising a top surface configured to face an inferior bony surface of the superior vertebral bone, a bottom surface configured to face a superior bony surface of the inferior vertebral bone;
a second implant sized to be at least partially received within the intervertebral disc space and comprising a top surface configured to face a bony surface of the superior vertebral bone and a bottom surface configured to face a bony surface of the inferior vertebral bone; and
a non-implantable placement assembly configured to advance the first implant and the second implant to at least one target location, the non-implantable placement assembly extending from a trailing end portion thereof to a leading end portion thereof via at least an elongate member, the trailing end portion comprising a handle;
wherein:
the first implant is configured to at least temporarily couple with the leading end portion of the non-implantable placement assembly; and
the second implant is configured to be at least partially advanced towards the leading end portion along a longitudinal axis of the elongate member.

22. The device assembly of claim 21, wherein the second implant comprises a cavity configured to receive at least a portion of the elongate member.

23. The device assembly of claim 21, wherein the non-implantable placement assembly further comprises a pusher configured to advance the second implant along the elongate member, at least one surface of the second implant abutting the elongate member when the second implant is coupled to the non-implantable placement assembly.

24. The device assembly of claim 23, wherein the non-implantable placement assembly is configured such that when the pusher is actuated, the second implant is advanced (i) for a first segment, along a direction at least parallel with a first longitudinal axis of the elongate member and (ii) for a second segment, along a direction oblique to the first longitudinal axis.

25. The device assembly of claim 23, wherein the first implant is configured to, when positioned at least partially within the intervertebral disc space, increase a distance between the inferior bony surface of the superior vertebral bone and the superior bony surface of the inferior vertebral bone.

26. The device assembly of claim 25, wherein a distance between the top surface and the bottom surface of the first implant is variable.

27. The device assembly of claim 21, wherein the second implant is configured to couple at least temporarily with the non-implantable placement assembly.

28. The device assembly of claim 21, wherein the first implant comprises a distraction mechanism configured to, when actuated, vary a distance between the top surface and the bottom surface of the first implant.

29. The device assembly of claim 28, wherein the distraction mechanism is configured to be actuated to vary the distance via a mechanism of the non-implantable placement assembly when the first implant is coupled to the non-implantable placement assembly.

30. The device assembly of claim 21, wherein the handle is disposed at least approximately orthogonal to the longitudinal axis of the elongate member.

31. The device assembly of claim 21, wherein the non-implantable placement assembly is rendered non-implantable at least by virtue of a size thereof, the size not being compatible with implantation in a human being.

32. Apparatus for treatment of a functional spinal unit of a living subject, the functional spinal unit comprising a superior vertebral bone, an inferior vertebral bone, and an intervertebral disc space positioned there between, the apparatus comprising:
a first implantable element sized to be at least partially received within the intervertebral disc space and comprising a top surface configured to face an inferior bony surface of the superior vertebral bone, a bottom surface configured to face a superior bony surface of the inferior vertebral bone;
a second implantable element sized to be at least partially received within the intervertebral disc space and comprising a top surface configured to face an inferior bony surface of the superior vertebral bone and a bottom surface configured to face a superior bony surface of the inferior vertebral bone; and
a placement assembly separable from both the first implantable element and the second implantable element, the placement assembly configured to advance the first implantable element and the second implantable element to at least one target location;
wherein the first implantable element is configured such that it can both (a) act as a mechanism capable of varying at least a distance between the inferior bony surface of the superior vertebral bone and the superior bony surface of the inferior vertebral bone, and (b) act as an implant to be retained within the intervertebral disc space after separation of the first implantable element and the second implantable element from the placement assembly.

33. The apparatus of claim 32, wherein the placement assembly is configured to place the first implantable element within the intervertebral disc space before placement of the second implantable element in the intervertebral disc space.

34. The apparatus of claim 33, wherein the placement assembly is further configured to, after said placement of the first implantable element within the intervertebral disc space, set the distance between the inferior bony surface of the superior vertebral bone and the superior bony surface of the inferior vertebral bone to a desired value via at least the varying, before placement of the second implantable element in the intervertebral disc space.

35. The apparatus of claim 34, wherein the desired value comprises a value which is based at least in part on at least one of a size or configuration of the second implantable element.

36. The apparatus of claim 32, wherein the placement assembly comprises an elongate track along which (i) the second implantable element can be advanced into the intervertebral disc space; and (ii) at least one third implantable element can be advanced into the intervertebral disc space.

37. The apparatus of claim 32, wherein the first implantable element comprises a mechanism which is actuatable via the placement assembly, the actuation of the mechanism configured to provide the varying of the at least distance.

38. The apparatus of claim 32, wherein:
the at least one target location comprises a first target location, and a second target location, the first and second target locations at least partly juxtaposed relative to one another; and
the placement assembly configured to advance the first implantable element and the second implantable element to the at least one target location comprises a placement assembly configured to (i) dispose the first implantable element to the first target location via a distal end of the placement assembly, and (ii) dispose the second implantable element to the second target location via at least a track which is, for at least a terminal portion of its length, offset from the distal end.

39. Apparatus for treatment of a functional spinal unit of a living subject, the functional spinal unit comprising a superior vertebral bone, an inferior vertebral bone, and an intervertebral disc space positioned there between, the apparatus comprising:
an implant placement instrument configured to dispose both a first, actuatable implantable element and a second implantable element within the intervertebral disc space, the implant placement instrument further configured to:
(i) dispose the first, actuatable implantable element at a first target location within the intervertebral disc space prior to disposition of the second implantable element at a second target location within the intervertebral disc space;
(ii) after disposition of the first, actuatable implantable element at the first target location, actuate the first, actuatable implantable element so as to achieve a desired relationship between the superior vertebral bone and the inferior vertebral bone; and
(iii) after the actuation, cause placement of the second implantable element at the second target location.

40. The apparatus of claim 39, wherein the implant placement instrument is further configured to selectively disengage the first, actuatable implantable element after the actuation.

41. The apparatus of claim 39, wherein the implant placement instrument is further configured to engage the first, actuatable implantable element prior to the disposition thereof at the first target location, and selectively disengage the first, actuatable implantable element after the actuation.

42. The apparatus of claim 41, further comprising engagement apparatus configured to provide said engagement of the first, actuatable implantable element prior to the disposition thereof at the first target location, and said selective disengagement of the first, actuatable implantable element after the actuation, responsive to operation of the engagement apparatus by a user of the implant placement instrument.

43. The apparatus of claim 41, wherein:
the engagement of the first, actuatable implantable element comprises maintenance of the first, actuatable implantable element in a given position relative to a distal portion of the implant placement instrument; and
the selective disengagement of the first, actuatable implantable element comprises complete release of the first, actuatable implantable element from the implant placement instrument.

44. The apparatus of claim 41, further comprising actuation apparatus configured to, responsive to rotational input of a user of the implant placement instrument, cause the actuation of the first, actuatable implant element.

45. The apparatus of claim 41, further comprising a track apparatus configured to releasably engage the second implantable element, the track apparatus further configured to guide a movement of the second implantable element toward the second target location when the implant placement instrument is positioned such that the first, actuatable implantable element is delivered to the first target location.

46. The apparatus of claim 41, further comprising the first, actuatable implantable element, and wherein:
the first, actuatable implantable element is configured to releasably engage the implant placement instrument, and at least (i) assume a first configuration whereby a height of the first, actuatable implantable element comprises a first value, and (ii) as part of said actuation, assume a second configuration whereby the height of the first, actuatable implantable element comprises a second value greater than the first value.

47. The apparatus of claim 46, further comprising the second implantable element;
wherein the second implantable element a) comprises a fixed geometry, and b) is configured to be received within an implant guidance apparatus of the implant placement instrument.
